(19) United States
(12) Patent Application Publication
(10) Pub. No.: US 2025/0381163 A1
(43) Pub. Date: Dec. 18, 2025

(54) Chelating Agents for Use in Cancer Therapy

(71) Applicant: Pleco Therapeutics B.V., Nijmegen (NL)

(72) Inventors: Dan Gelvan, Modi'ln (IL); Patricia Muller, Durham (GB)

(21) Appl. No.: 18/867,555

(22) PCT Filed: May 23, 2023
(86) PCT No.: PCT/NL2023/050297

§ 371 Date: Nov. 20, 2024

(30) Foreign Application Priority Data
May 23, 2022 (NL) 2031966

(51) Int. Cl. A61K 31/225; A61K 31/194; A61K 45/06; A61P 35/00; A61P 39/04

(52) U.S. Cl. CPC A61K 31/225; A61K 31/194; A61K 45/06; A61P 35/00; A61P 39/04

(57) ABSTRACT

The invention provides a chelating agent for use in a method of treating cancer in a subject, wherein said cancer has a resistance to an anti-cancer therapeutic agent. Further, the invention provides a chelating agent for use in a method of sensitizing a subject for an anti-cancer treatment and/or counteracting a resistance to an anti-cancer therapeutic agent, wherein the method restores or re-activates a tumor suppressor protein function that preferably was impaired due to aberrant tumor suppressor protein folding. The invention also provides a pharmaceutical composition comprising a 2,3-Dimercapto-1-propanesulfonic acid (DMPS) or DMSA, preferably miaDMSA, and a pharmaceutically acceptable excipient; wherein the DMPS is present in a dose of 40-12000 mg.

### FIELD OF THE INVENTION

The present invention relates to the field of cancer therapy. More particularly, the invention relates to the use of a chelating agent to reverse a resistance of a cancer towards one or more anti-cancer therapeutic agents.

### BACKGROUND OF THE INVENTION

It is well established that the tumor suppressor p53 is required for both the prevention of cancer and the tumor cell death upon chemotherapy. However, the central gatekeeping functions of p53 are known to be compromised by elevated levels of several metals (e.g., Cu, Pb, Cd, As and others) that cause loss of its protective function and potentiation of aggressive tumor invasiveness. Such metals are found elevated in multiple cancer types.

Mutated p53 often loses its tumor suppressor and apoptosis inducer function, while it may gain one or more of a different subset of functions that make the tumor more aggressive, more metastatic and resistant to chemotherapy, termed gain-of-function. Loss of function and gain of function of p53 is mediated by an alternative protein folding such as a misfolding or unfolding. Said alternative folding can be caused by mutations in the p53 gene, but can also be attributed to environmental factors, such as the presence of elevated levels of certain metals, or combinations of p53 mutations and environmental factors such as elevated levels of certain metals. In the literature, zinc is reported to be required for proper p53 folding, while it is suggested that iron, copper, lead, mercury, cadmium, nickel, arsenic as well as vanadium are suspected to serve as drivers of aberrant p53 folding (Muller et al., Toxicology Research, 4:3, p. 576-591 (2015)).

In cancer therapy, therapeutic agents, such as chemotherapeutic agents, are commonly employed to halt tumor growth and/or reduce tumor size. However, tumors often have or develop a resistance against a therapeutic agent such that the patient will no longer sufficiently respond to said therapy, leading to the disability to effectively treat patients suffering from cancer. As examples, small cell lung cancer (SCLC), and AML, respond poorly to chemotherapy. AML is an example of a cancer that is associated with elevated levels of metals including, amongst others, arsenic, copper, cadmium, nickel and chromium as measured in serum.

There is a need in the art for methods that counteract or alleviate a resistance of a cancer towards anti-cancer therapeutic agents. In other words, there is a need for a therapy that allows for the treatment of a subject with a resistant cancer, i.e., a cancer which has developed a resistance to one or more anti-cancer therapeutic agents, and wherein said resistant cancer is to be sensitized towards treatment with said anti-cancer therapeutic agent by reducing or reversing its resistance to the anti-cancer therapeutic agents.

### SUMMARY OF THE INVENTION

Unexpectedly, the inventors have discovered that a chelating agent can be successfully employed to reverse a chemoresistance of a resistant cancer. Without wishing to be bound by theory, it is hypothesized that this form of sensitization of the resistant cancer through chelation therapy, such as multi-metal chelation therapy, results in restoration of function of tumor suppressors, such as p53, which subsequently allows for, or restores, the induction of tumor cell death in response to treatment with the chemotherapeutic agent to which the cancer was resistant. The Examples and Figures herein below show, inter alia, that (i) a chelating agent can sensitize a tumor having a metal-induced chemoresistance towards treatment with the chemotherapeutic agent to which the cancer showed to be resistant, (ii) that wild-type tumor suppressor protein p53 is unfolded under conditions of elevated metal levels, and (iii) that restoration of chemosensitivity is more pronounced in those cells that express a p53 that can re-fold to a native-like conformation of wild-type p53 (e.g. wild-type p53 and mutants thereof that can re-fold back to the native conformation of wild-type p53).

Therefore, the invention provides in a first aspect a chelating agent for use in a method of treating a cancer in a subject, wherein said cancer has a resistance to an anti-cancer therapeutic agent. The invention also provides a chelating agent for use in a method of treating a cancer in a subject, wherein said chelating agent is for use in a method of restoring sensitivity of said cancer to an anti-cancer therapeutic agent. Preferably, said method provides for restoration or re-activation of tumor suppressor protein function; more preferably wherein, prior to administration of said chelating agent, said tumor suppressor protein function was impaired as a result of (or due to) aberrant tumor suppressor protein folding. Further, the invention provides a chelating agent for use in a method of sensitizing a subject for an anti-cancer treatment (e.g. sensitizing a subject for a treatment with an anti-cancer therapeutic agent) and/or counteracting a resistance to an anti-cancer therapeutic agent, wherein the method (effectively) restores a tumor suppressor protein function which preferably was impaired as a result of (or due to) aberrant tumor suppressor protein folding. In other words, the invention provides a chelating agent for use in a method of sensitizing a subject for an anti-cancer treatment (e.g. sensitizing a subject for a treatment with an anti-cancer therapeutic agent) and/or counteracting a resistance to an anti-cancer therapeutic agent, wherein the method provides for restoration or re-activation of tumor suppressor protein function; more preferably wherein, prior to administration of said chelating agent, said tumor suppressor protein function was impaired as a result of aberrant tumor suppressor protein folding.

In a preferred embodiment of a medical use of the invention, said chelating agent is for use in a method of counteracting a resistance of said cancer to said anti-cancer therapeutic agent.

In another preferred embodiment of a medical use of the invention, said chelating agent is for use in a method of counteracting a resistance of said cancer to said anti-cancer therapeutic agent, wherein said resistance is caused by cancer cells having an impaired tumor suppressor protein function.

In another preferred embodiment of a medical use of the invention, said chelating agent is for use in restoration or re-activation of tumor suppressor protein function.

In another preferred embodiment of a medical use of the invention, said chelating agent is administered prior to administration of said anti-cancer therapeutic agent.

In another preferred embodiment of a medical use of the invention, said chelating agent is not administered together with the anti-cancer therapeutic agent at the same time (i.e., in the absence of an anti-cancer therapeutic agent). Instead the anti-cancer therapeutic agent is preferably administered (for instance at least 10 minutes, at least 30 minutes, at least one hour, at least 12 hours or at least 24 hours) after the chelating agent has been administered. This can also be referred to as “solitarily” administration of the chelating agent, i.e., not together at the same time with an anti-cancer therapeutic agent, but in the absence of an anti-cancer therapeutic agent.

In another preferred embodiment of a medical use of the invention, said chelating agent is administered prior to administration of said anti-cancer therapeutic agent, preferably wherein the chelating agent is administered prior to administration of said anti-cancer therapeutic agent (in order) to restore or re-activate tumor suppressor protein function, more preferably wherein the chelating agent is administered at least 10 minutes, at least 30 minutes, at least one hour or at least three hours prior to administration of said anti-cancer therapeutic agent to restore or re-activate tumor suppressor protein function, most preferably wherein the chelating agent is administered at least 10 minutes, at least 30 minutes or at least one hour prior to a first, second, third and/or every continuing administration cycle of said anti-cancer therapeutic agent to restore or re-activate tumor suppressor protein function.

In another preferred embodiment of a medical use of the invention, said chelating agent is solitarily administered, preferably for at least 10 minutes, at least 30 minutes, at least one hour, at least 12 hours, or at least 24 hours prior and/or after an anti-cancer therapeutic agent or a combination of an anti-cancer therapeutic agent and a chelating agent is administered.

In another preferred embodiment of a medical use of the invention, said chelating agent is for administration in combination with said anti-cancer therapeutic agent.

In another preferred embodiment of a medical use of the invention, said resistance is a chemoresistance; and said anti-cancer therapeutic agent is a chemotherapeutic agent.

In another preferred embodiment of a medical use of the invention, said cancer cells of said cancer have an impaired tumor suppressor protein function.

In another preferred embodiment of a medical use of the invention, said impaired tumor suppressor protein function is the result of aberrant tumor suppressor protein folding.

In another preferred embodiment of a medical use of the invention, said cancer cells of said cancer comprise an aberrantly folded tumor suppressor protein resulting in impaired tumor suppressor protein function.

In another preferred embodiment of a medical use of the invention, said tumor suppressor protein is one or more tumor suppressor proteins selected from the group consisting of p53, p63 and p73. In another preferred embodiment of a medical use of the invention, said tumor suppressor protein is p53; and preferably said tumor suppressor protein function is one or more selected from the group formed by: negative regulation of the cell cycle, and promotion of apoptosis.

In another preferred embodiment of a medical use of the invention, said p53 is a wild-type p53 or a mutated p53.

In another preferred embodiment of a medical use of the invention, said chelating agent is for use in a method of counteracting a resistance of said cancer to said anti-cancer therapeutic agent, wherein said resistance is caused by cancer cells having an impaired p53 protein function, preferably an impaired wildtype p53 protein function and/or impaired mutant p53 protein function.

In another preferred embodiment of a medical use of the invention, said chelating agent is for use in a method of counteracting a resistance of said cancer to said anti-cancer therapeutic agent, wherein said resistance is caused by cancer cells having an aberrant p53 protein folding, preferably aberrant wildtype p53 protein folding and/or aberrant mutant p53 protein folding.

In another preferred embodiment of a medical use of the invention, said chelating agent is for use in a method of counteracting a resistance of said cancer to said anti-cancer therapeutic agent, wherein said resistance is caused by cancer cells having an aberrant p53 protein function, preferably aberrant wildtype p53 protein function and/or aberrant mutant p53 protein folding, wherein said chelating agent is solitarily administered, i.e., in the absence of an anti-cancer therapeutic agent, for example for at least 10 minutes, at least 30 minutes, at least one hour, at least 12 hours, or at least 24 hours prior and/or after an anti-cancer therapeutic agent or a combination of an anti-cancer therapeutic agent and a chelating agent is administered; preferably wherein said chelating agent is solitarily administered, i.e., in the absence of an anti-cancer therapeutic agent, for example for at least 10 minutes, at least 30 minutes or at least one hour prior and/or after a first, second, third or every continuing administration cycle of said anti-cancer therapeutic agent and/or of a combination of said anti-cancer therapeutic agent and a chelating agent is administered. In another preferred embodiment of a medical use of the invention, said cancer is characterized by the presence of at least two, more preferably at least 3, 4, 5, 6, 7, 8, 9, 10, 11, 12, 13, 14, 15, 16, 17, 18, 19, 20, 21, 22 or at least 23 metals selected from the group consisting of arsenic (As), aluminum (Al), antimony (Sb), Barium (Ba), boron (B), cadmium (Cd), Cerium (Ce), Chromium (Cr), lead (Pb), mercury (Hg), neodymium (Nd), manganese (Mn), Nickel (Ni), tin (Sn), titanium (Ti), uranium (U), vanadium (V), copper (Cu), iron (Fe), gold (Au), silver (Ag), palladium (Pd) and platinum (Pt).

In another preferred embodiment of a medical use of the invention, said cancer is characterized by the presence of elevated levels (e.g. elevated levels inside cancer cells) of at least two, more preferably at least 3, 4, 5, 6, 7, 8, 9, 10, 11, 12 13, 14, 15, 16, 17, 18, 19, 20, 21, 22 or at least 23 metals selected from the group consisting of arsenic (As), aluminum (Al), antimony (Sb), Barium (Ba), boron (B), cadmium (Cd), Cerium (Ce), Chromium (Cr), lead (Pb), mercury (Hg), neodymium (Nd), manganese (Mn), Nickel (Ni), tin (Sn), titanium (Ti), uranium (U), vanadium (V), copper (Cu), iron (Fe), gold (Au), silver (Ag), palladium (Pd) and platinum (Pt). An elevated level of metals includes levels that are at least 1.1, more preferably at least 1.2, 1.5, 2, 3, 4, 5, or at least 10 times higher than the level of metals in a suitable control, such as a level that can be measured in cancers of the same cancer type that are not resistant to the anti-cancer therapeutic agent, and preferably in which the function of tumor suppressor proteins is normal, e.g, wherein tumor suppressor proteins, such as p53, are in their native conformation (folding).

In another preferred embodiment of a medical use of the invention, said cancer is characterized by the presence of elevated levels (e.g. elevated levels inside cancer cells) of at least one, preferably at least two, more preferably at least 3, 4, 5, 6, 7, 8 or at least 9 metals selected from the group consisting of copper (Cu), iron (Fe), lead (Pb), mercury (Hg), cadmium (Cd), Nickel (Ni), arsenic (As), vanadium (V) and Chromium (Cr).

In another preferred embodiment of a medical use of the invention, said cancer is characterized by the presence of (e.g. elevated levels of) at least one, preferably at least two, more preferably at least 3, 4, 5 or at least 6 metals selected from the group consisting of chromium (Cr), manganese (Mn), copper (Cu), cadmium (Cd), mercury (Hg) and lead (Pb).

In another preferred embodiment of a medical use of the invention, said aberrant tumor suppressor protein folding is induced by elevated levels of metals as defined in any one of the previous embodiments.

In another preferred embodiment of a medical use of the invention, said method of treating a cancer is a method of chemosensitizing a cancer of a subject.

In another preferred embodiment of a medical use of the invention, said method of treating a cancer is a method of potentiating an anti-cancer effect of said anti-cancer therapeutic agent.

In another preferred embodiment of a medical use of the invention, said anti-cancer effect that is potentiated is selected from the group consisting of a cytotoxic effect, a cytostatic effect, anti-invasiveness, anti-dissociation, anti-vascularization and combinations thereof.

In another preferred embodiment of a medical use of the invention, said resistance to said anti-cancer therapeutic agent is a metal-induced resistance, preferably wherein said metal-induced resistance is the result of the presence of at least two, more preferably at least 3, 4, 5, 6, 7, 8, 9, 10, 11, 12, 13, 14, 15, 16, 17, 18, 19, 20, 21, 22 or at least 23 metals selected from the group consisting of arsenic (As), aluminum (Al), antimony (Sb), Barium (Ba), boron (B), cadmium (Cd), Cerium (Ce), Chromium (Cr), lead (Pb), mercury (Hg), neodymium (Nd), manganese (Mn), Nickel (Ni), tin (Sn), titanium (Ti), uranium (U), vanadium (V), copper (Cu), iron (Fe), gold (Au), silver (Ag), palladium (Pd) and platinum (Pt).

In another preferred embodiment of a medical use of the invention, said resistance to said anti-cancer therapeutic agent is associated with, mediated by or the result of metal-induced aberrant folding of p53 protein in a cancer cell.

In another preferred embodiment of a medical use of the invention, said anti-cancer therapeutic agent is an anthracycline, more preferably doxorubicin; an antimetabolite, more preferably 5-fluorouracil (5-FU); and/or a taxane, more preferably Nab-paclitaxel and/or Paclitaxel.

In another preferred embodiment of a medical use of the invention, said chelating agent is 2,3-dimercaptosuccinic acid (DMSA), preferably monoisoamylDMSA (miaDMSA), DMPS and/or EDTA.

In another preferred embodiment of a medical use of the invention, said chelating agent is administered in combination with a second chelating agent.

In another preferred embodiment of a medical use of the invention, (i) said chelating agent is a 2,3-dimercapto-1-propanesulfonic acid (DMPS) and optionally wherein said second chelating agent, if present, is an EDTA; or (ii) said chelating agent is a 2,3-dimercaptosuccinic acid (DMSA), preferably monoisoamylDMSA (miaDMSA), and optionally wherein said second chelating agent, if present, is an EDTA.

In another preferred embodiment of a medical use of the invention, said chelating agent and optionally said second chelating agent are provided in the form of a fixed-dose product (preferably a fixed dose combination product), such as (i) a fixed-dose pharmaceutical composition comprising said chelating agent and optionally said second chelating agent or (ii) a fixed-dose kit comprising a first container that comprises said chelating agent and a second container that comprises said second chelating agent.

In another preferred embodiment of a medical use of the invention, said chelating agent, and optionally said second chelating agent, are administered parenterally (preferably intravenously or intratumorally) or enterally (preferably orally or rectally). In embodiments, said (first) chelating agent and said second chelating agent are administered via the same route of administration or via a different route of administration.

In another preferred embodiment of a medical use of the invention, said anti-cancer therapeutic agent is administered parenterally such as intravenously or intratumorally.

In another preferred embodiment of a medical use of the invention, said chelating agent, and optionally said second chelating agent, is/are administered in a dose of 1-100 mg/kg body weight/day, daily for 1-25 days of each cycle, and provided in repeated cycles at intervals (e.g. intervals typically 3-6 weeks apart).

In another preferred embodiment of a medical use of the invention, said cancer is solid tumor or a liquid tumor.

In another preferred embodiment of a medical use of the invention, said cancer is a breast cancer, a lung cancer such as small cell lung cancer (SCLC), a pancreatic cancer or a blood cancer such as acute myeloid leukemia (AML).

In another aspect, the invention provides a pharmaceutical composition comprising a 2,3-Dimercapto-1-propanesulfonic acid (DMPS) and a pharmaceutically acceptable excipient; wherein the DMPS is present in a dose of 40-12000 mg, for instance 40-6000 mg, 100-5000 mg, 200-4000 mg or 400-3600 mg; preferably wherein said composition is for daily administration.

In another aspect, the invention provides a pharmaceutical composition comprising (i) a 2,3-Dimercapto-1-propanesulfonic acid (DMPS) or a DMSA, preferably miaDMSA, (ii) an EDTA, and (iii) a pharmaceutically acceptable excipient; preferably wherein said DMPS or said DMSA is present in a dose of instance 40-6000 mg, 100-5000 mg, 200-4000 mg or 400-3600 mg.

In a preferred embodiment of a pharmaceutical composition of the invention, the composition further comprises an anti-cancer therapeutic agent, preferably a chemotherapeutic agent, more preferably an anthracycline, most preferably doxorubicin.

In another aspect, the invention provides a pharmaceutical combination comprising (i) a first container comprising a pharmaceutical composition comprising a 2,3-Dimercapto-1-propanesulfonic acid (DMPS) or a DMSA, preferably miaDMSA, and a pharmaceutically acceptable excipient; and (ii) a second container comprising a pharmaceutical composition comprising an anti-cancer therapeutic agent, preferably a chemotherapeutic agent, more preferably an anthracycline such as doxorubicin, an antimetabolite, such as 5-fluorouracil (5-FU), and/or a taxane, such as Nab-paclitaxel and/or Paclitaxel, and a pharmaceutically acceptable excipient; and optionally wherein said combination comprises a third container comprising an EDTA, and a pharmaceutically acceptable excipient.

The invention also provides a method of treating a cancer in a subject, wherein said cancer has a resistance to an anti-cancer therapeutic agent, comprising the step of: —administering a therapeutically effective amount of a chelating agent to said subject. The invention also provides a method for restoring chemosensitivity in a subject having a cancer that is (at least partially) insensitive or resistant to chemotherapy, comprising the step of: administering a therapeutically effective amount of a chelating agent to said subject.

In preferred embodiments, said method provides for restoration or re-activation of tumor suppressor protein function; more preferably wherein, prior to administration of said chelating agent, said tumor suppressor protein function was impaired as a result of (or due to) aberrant tumor suppressor protein folding.

The invention also provides a method of sensitizing a subject for an anti-cancer treatment (e.g. sensitizing a subject for a treatment with an anti-cancer therapeutic agent) and/or counteracting a resistance to an anti-cancer therapeutic agent, comprising the step of: —administering a therapeutically effective amount of a chelating agent to said subject. In preferred embodiments, the method (effectively) restores a tumor suppressor protein function which preferably was impaired as a result of (or due to) aberrant tumor suppressor protein folding. In other words, the invention provides a method of sensitizing a subject for an anti-cancer treatment (e.g. sensitizing a subject for a treatment with an anti-cancer therapeutic agent) and/or counteracting a resistance to an anti-cancer therapeutic agent, wherein the method provides for restoration or re-activation of tumor suppressor protein function; more preferably wherein, prior to administration of said chelating agent, said tumor suppressor protein function was impaired as a result of aberrant tumor suppressor protein folding.

In preferred embodiments of said method of treating of the invention or said method of sensitizing and/or counteracting of the invention, said chelating agent is for administration in combination with said anti-cancer therapeutic agent (to which the cancer is resistant or insensitive).

All embodiments and/or aspects described in relation to a medical use of the invention also apply in relation to a method of treatment or method of sensitizing and/or counteracting of the invention (which is a medical use of the invention).

The invention also provides a use of a chelating agent for the manufacture of a medicament for treating a cancer in a subject; wherein said cancer has a resistance to an anti-cancer therapeutic agent. The invention also provides a use of a chelating agent for the manufacture of a medicament for restoring chemosensitivity in a subject having a cancer that is (at least partially) insensitive or resistant to chemotherapy.

In preferred embodiments of said use of a chelating agent of the invention, said chelating agent is for administration in combination with said anti-cancer therapeutic agent.

In another aspect, the invention provides a monoisoamylDMSA (miaDMSA) for use in a method of treating a cancer in a subject. Preferably, said miaDMSA is for administration in combination with an anti-cancer therapeutic agent as described herein, preferably a chemotherapeutic agent as described herein. Said cancer can be a solid tumor and/or a liquid tumor. Preferably, said miaDMSA is administered in the absence of said anti-cancer therapeutic agent, for example for at least 10 minutes, at least 30 minutes or at least one hour, at least 12 hours or at least 24 hours prior and/or after a first, second, third or every continuing administration cycle of said anti-cancer therapeutic agent and/or of a combination of said anti-cancer therapeutic agent and said miaDMSA is administered.

All embodiments and/or aspects described in relation to a medical use of the invention or method of treatment of the invention also apply in relation to a use of a chelating agent of the invention (which is a medical use of the invention).

In preferred embodiments, the pharmaceutical compositions or pharmaceutical combinations as disclosed herein are for use in the medical methods/uses of the invention.

### DESCRIPTION OF THE DRAWINGS

FIG. 1. Effect of metals on doxorubicin toxicity in Beas-2B cells.

Beas-2B lung cells were subjected to various concentration of a metal mix (mix consisting of chromium, manganese, zinc, copper, lead, mercury and cadmium (Table 1). A representative chemotherapeutic agent, doxorubicin, was added as indicated. Chemotherapy-induced cell death decreased in metal-treated cells in a dose-dependent manner thereby evidencing a metal-induced resistance towards chemotherapy.

FIG. 2. Effect of metals on p53 folding.

A. Western blot of the folding state of p53 from cells exposed to metal concentrations described in FIG. 1. When metal doses increase, protein unfolding increases, as indicated by the binding to the Ab240, an antibody specific for unfolded p53. B) Quantification (arbitrary units) of Ab240 binding in the Western blot.

FIG. 3. Effect of metal chelation on chemosensitivity.

The addition of a representative chelating agent, DMPS (150 mM), reversed the chemoresistance completely, restoring full chemosensitivity to three different cell types. The addition of chelators to cells not supplemented with metals had no effect, showing that the sensitization of cells is linked to the removal of excess metals and not an effect on the metals inherently present in the cell.
A. Effect of chelation on chemosensitivity in Beas-2B lung cells.
B. Effect of chelation on chemosensitivity in MCF7 breast cancer cells.
C. Effect of chelation on chemosensitivity in A549 lung cancer cells.
D. p53 dependence of chemoresistance and chelation restoration therapies

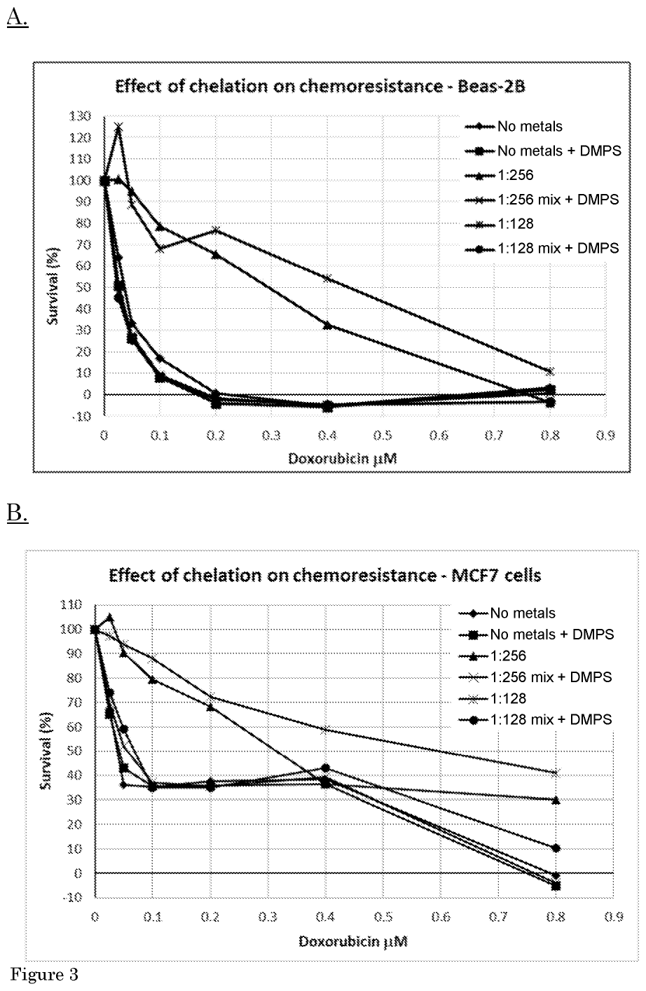

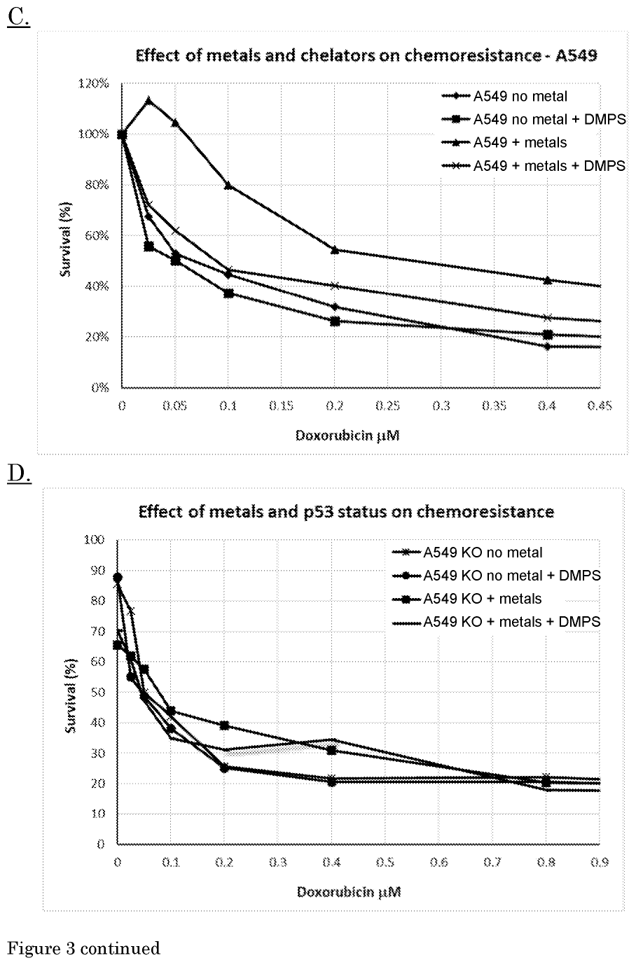

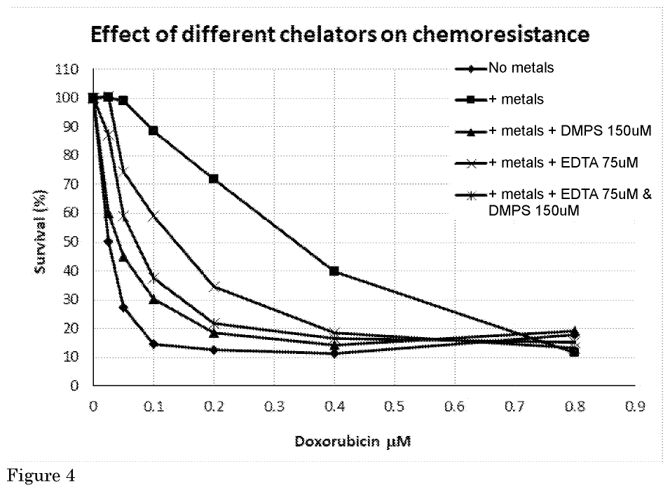

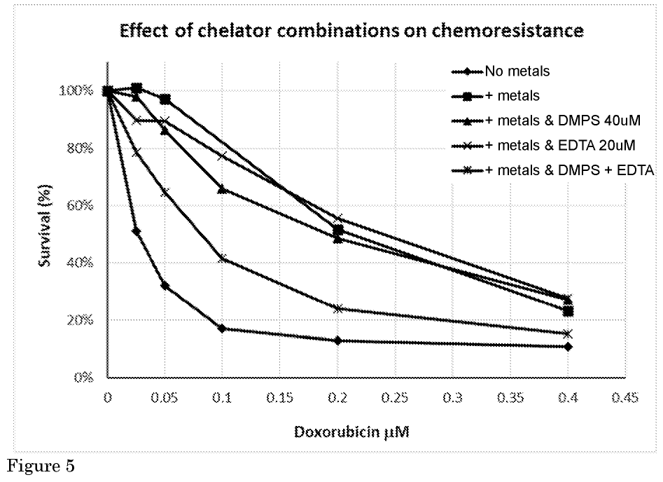

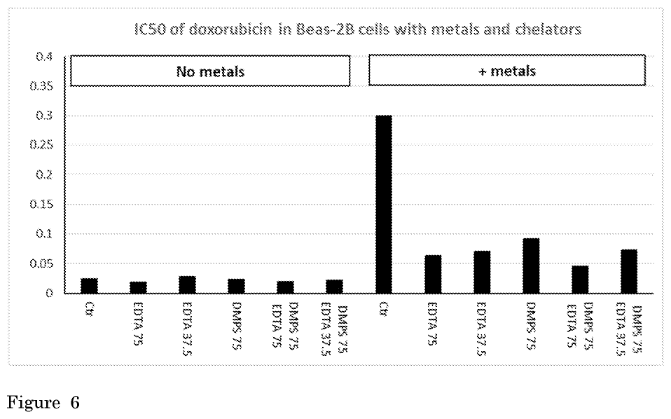

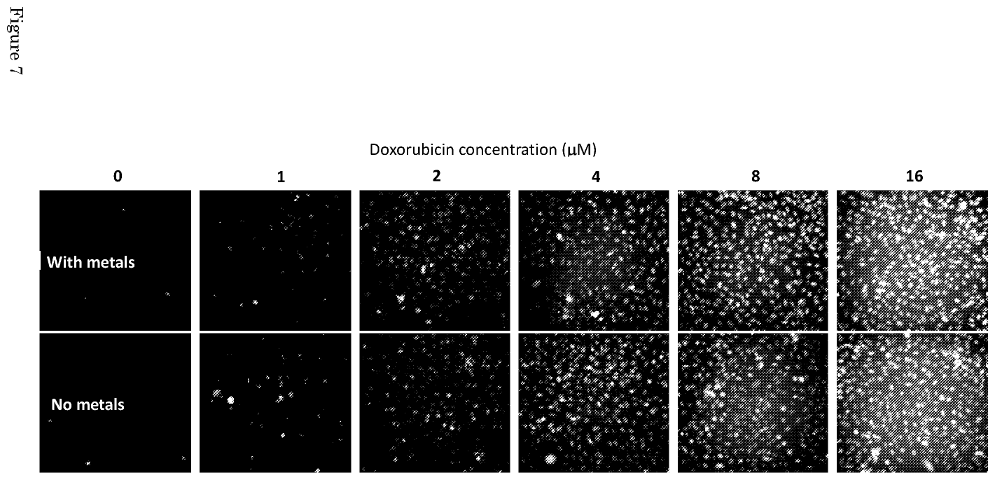

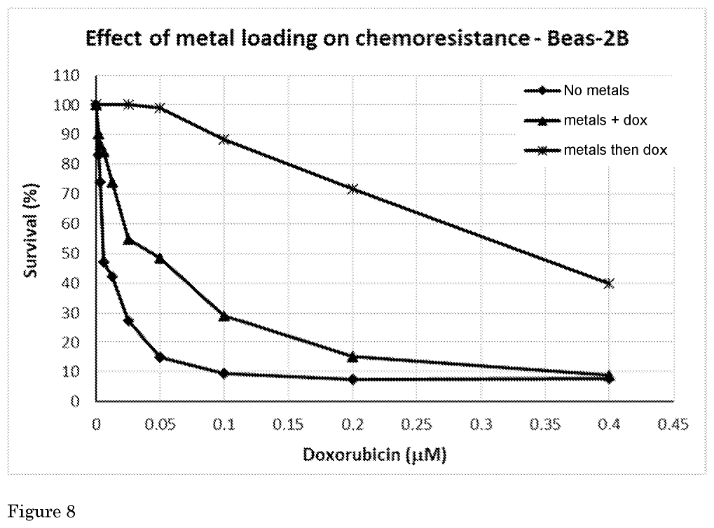

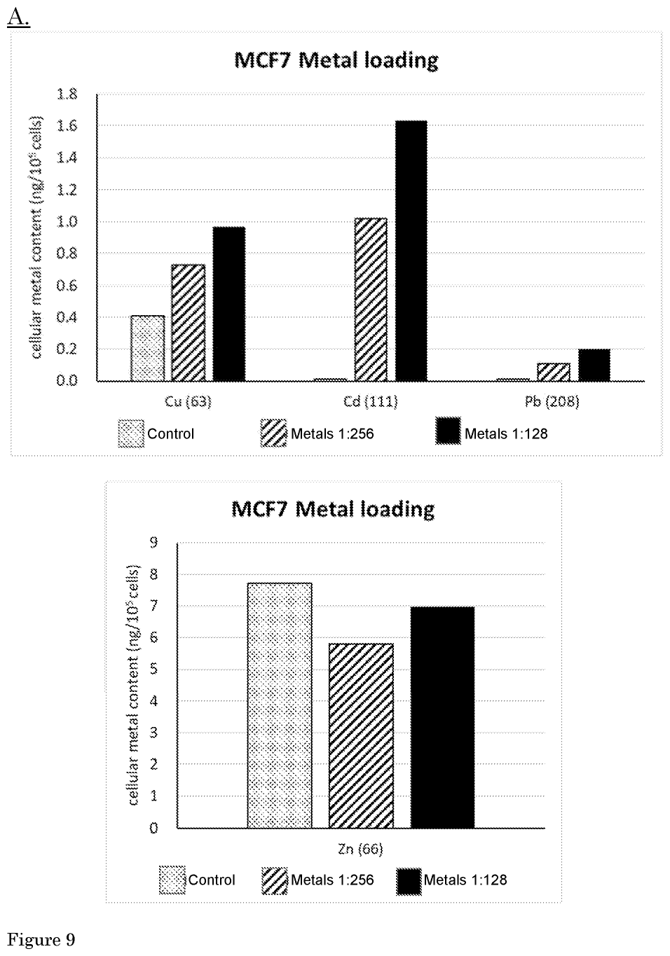

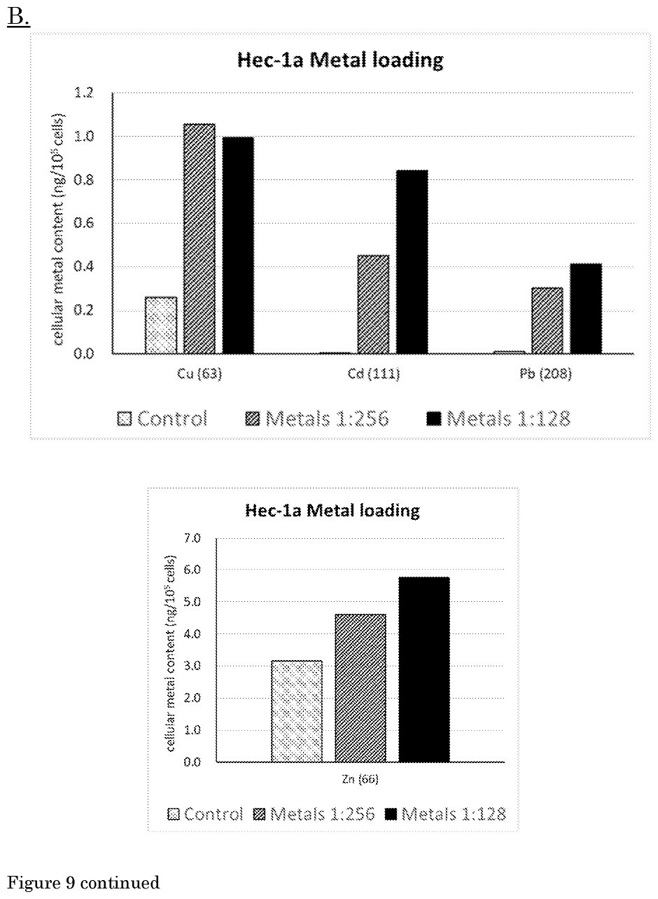

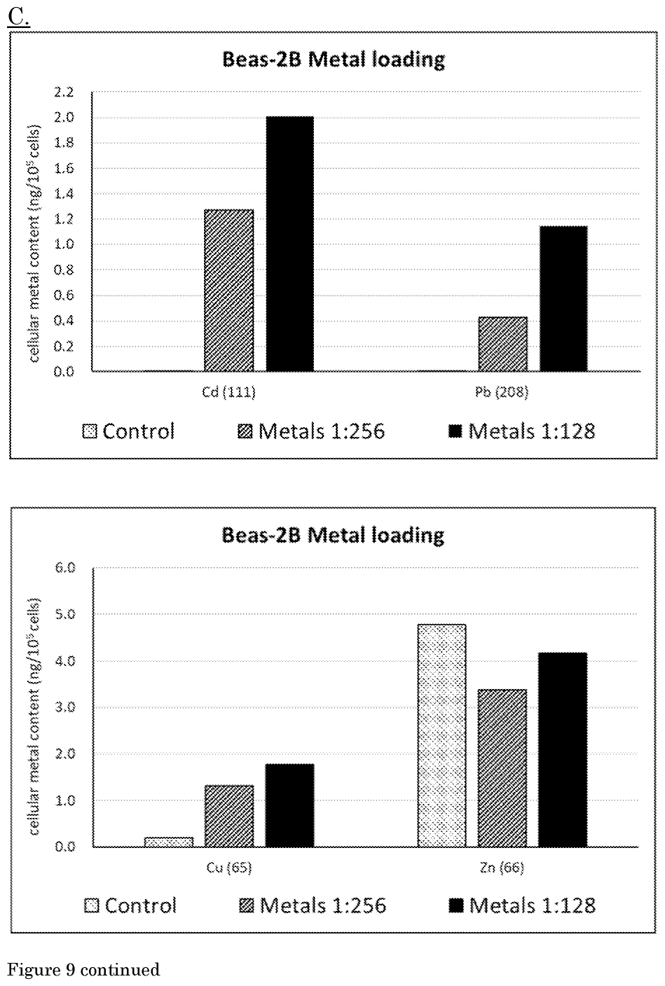

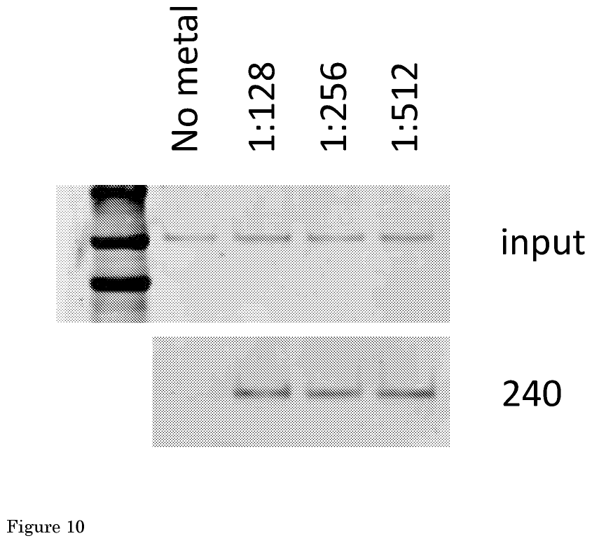

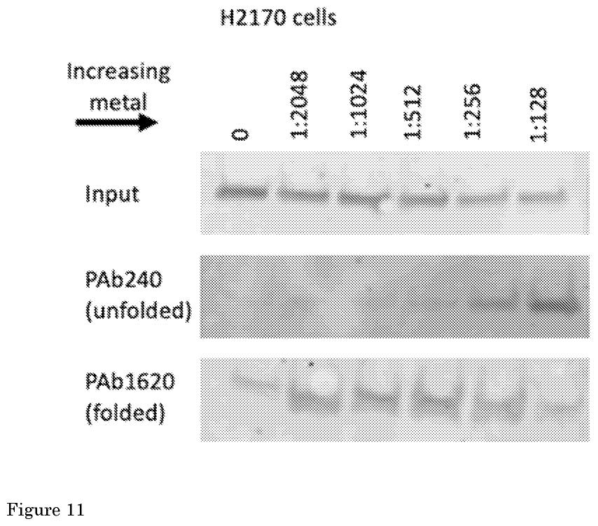

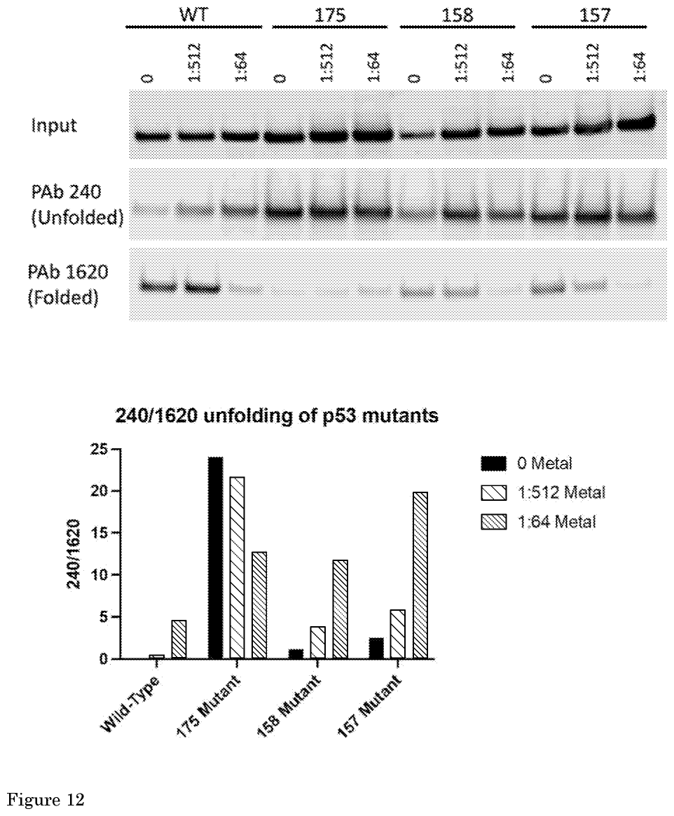

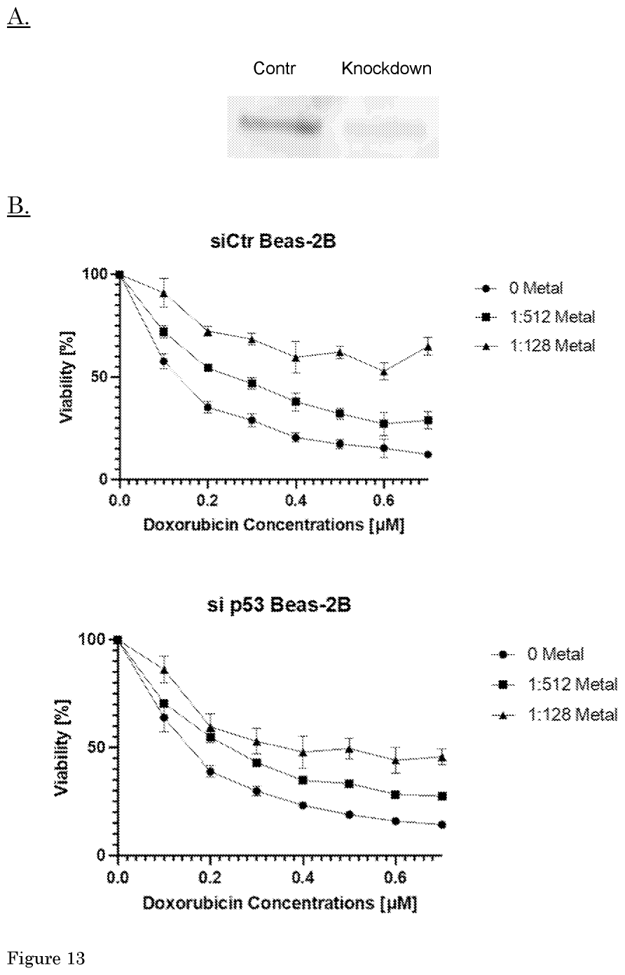

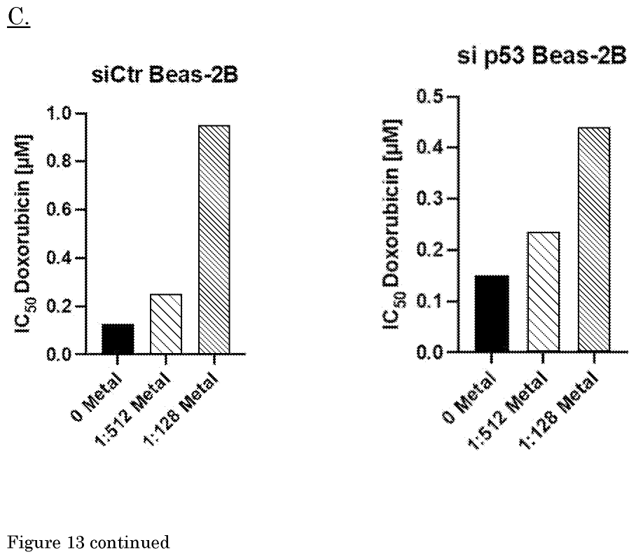

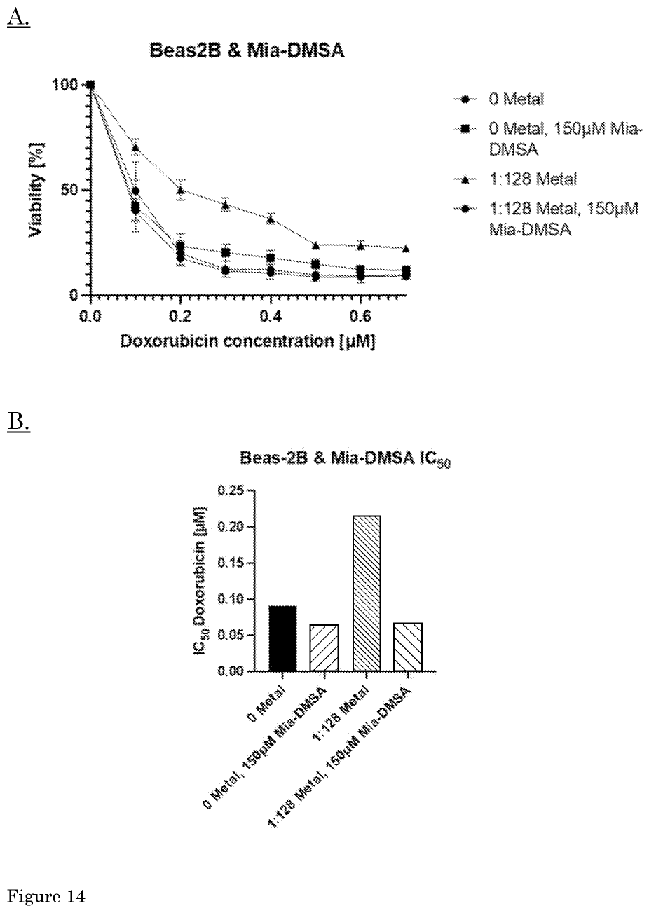

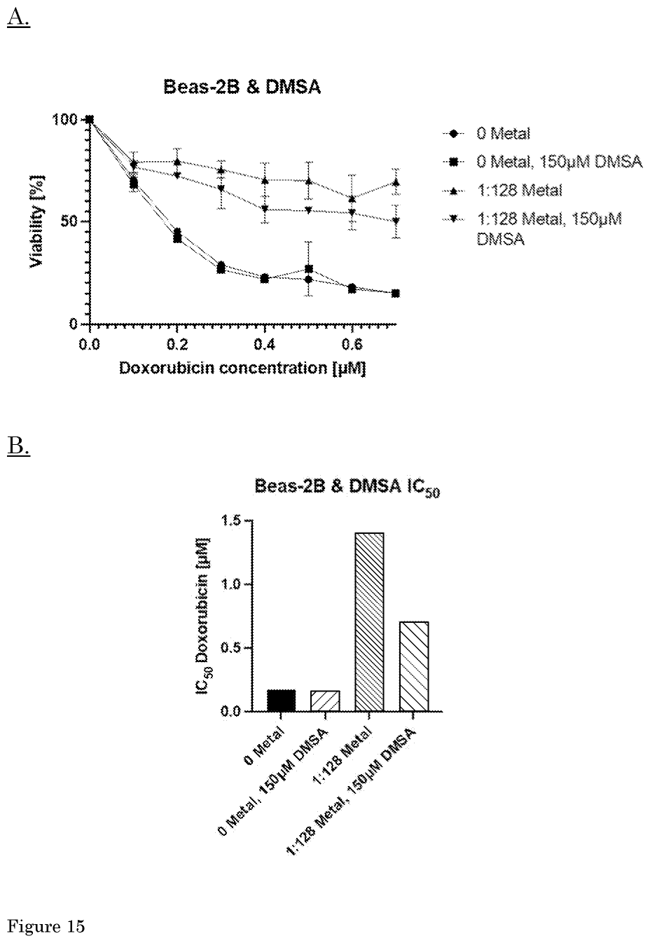

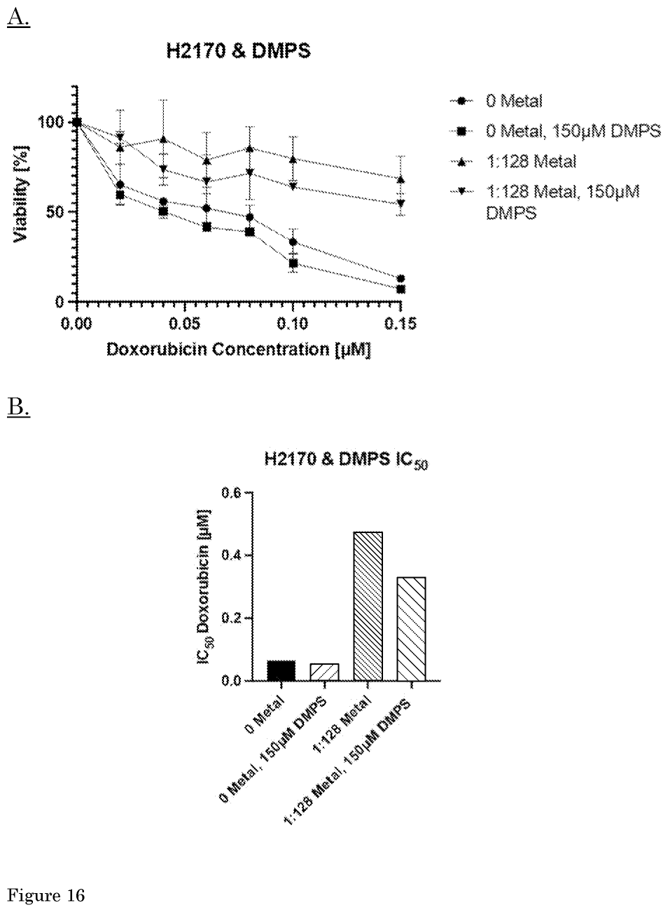

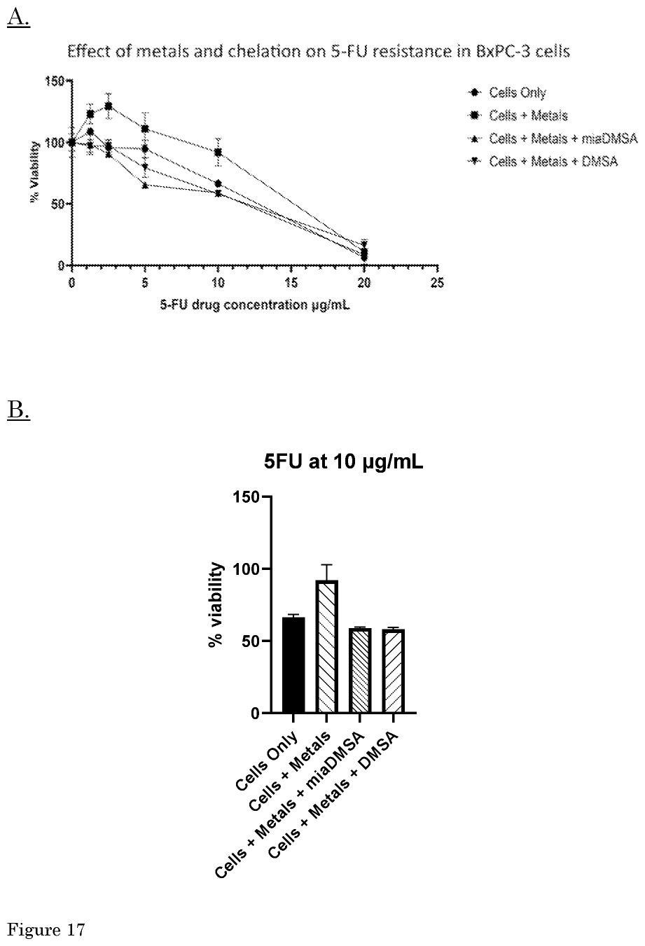

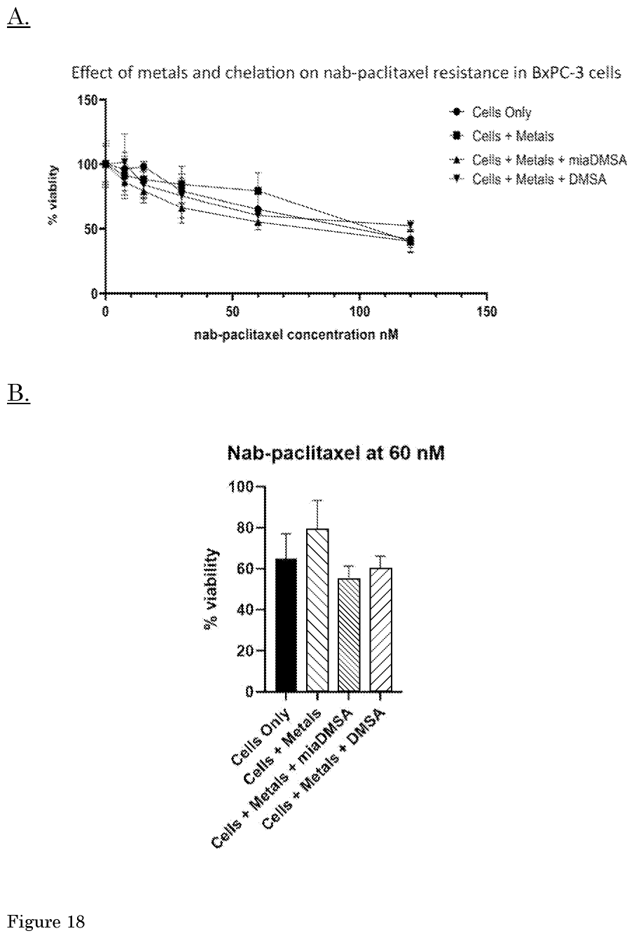

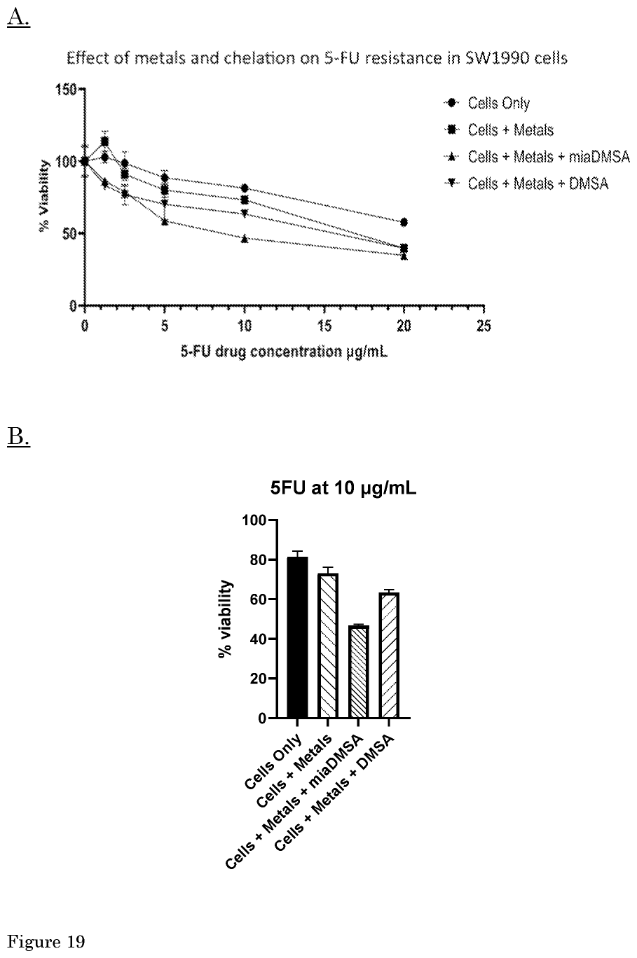

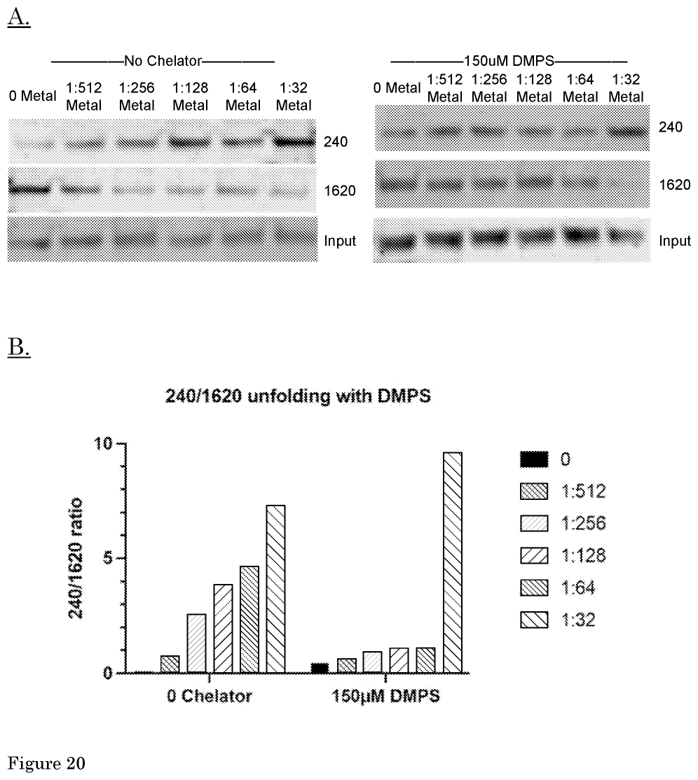

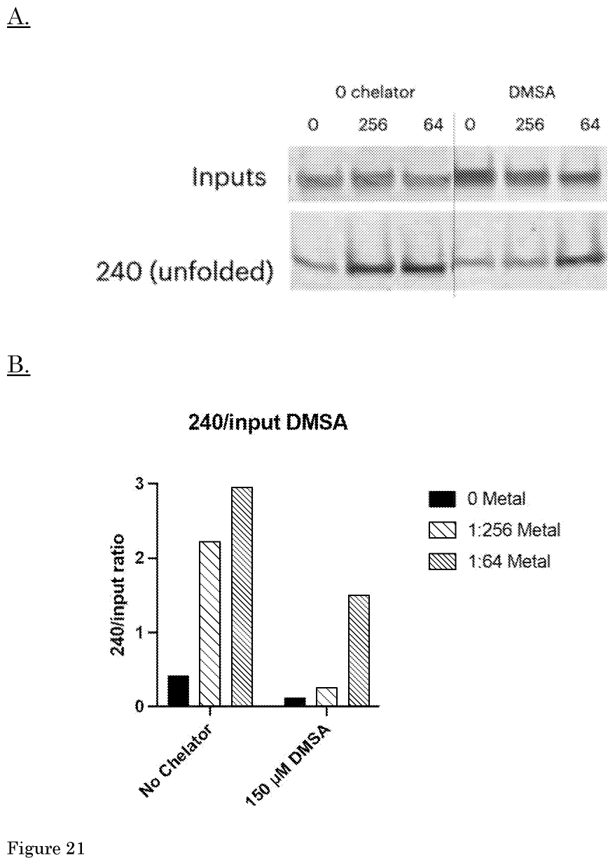

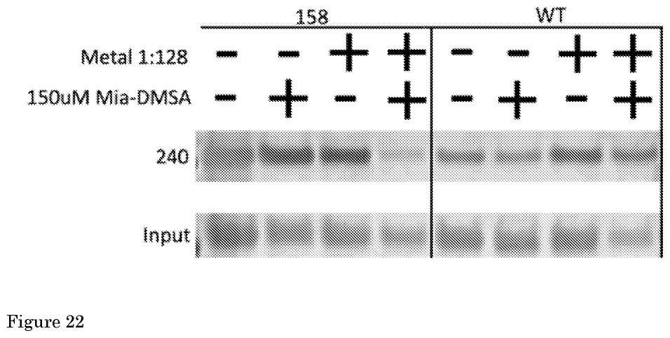

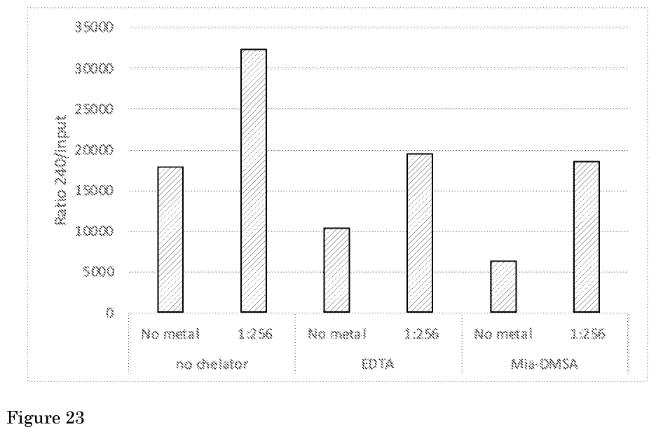

To demonstrate the p53 dependence of metal induced chemoresistance and its reversal by chelators, the effects of metals and DMPS (150 μM) on sensitivity to doxorubicin was tested in a p53 KO clone of A549. It was shown that metal induced chemoresistance and its reversal by DMPS were less pronounced than in native A549 cells (FIG. 3C).

FIG. 4. Effect of different chelators on restoration of chemosensitivity. The addition of chelating agents reversed the chemoresistance differentially. DMPS reversed chemoresistance completely whereas EDTA (provided in a dose providing equivalent binding capacity) reversed the resistance only partially in Beas-2B cells. Their combination also provided full reversal. Chelators had little effect on cells not loaded with metals.

FIG. 5. Chelators at non-saturating dose.

When the chelators were given at a lower, non-saturating, dose, a reinforcing effect was observed where each chelator by itself had only a small effect but reinforced each other when combined in Beas-2B cells. Without being bound by theory, the combined effect of chelators with different binding profiles may indicate that the chemoresistance is related to the overall metal load (a multi-metal toxicity) rather than to individual metals (single metal toxicity).

FIG. 6: IC50 of doxorubicin in cells treated with metals and chelators

Beas-2B cells, incubated either with or without metals, were treated with EDTA, DMPS or their combination as indicated in the Figure, and exposed to varying concentrations of doxorubicin. Survival was measured and the IC50 (the concentration of doxorubicin that killed 50% of the cells) was calculated. Metals had a major effect on the sensitivity of the cells to doxorubicin, inducing strong resistance. Chelators had no effect on the sensitivity of native cells to doxorubicin by largely reversed the sensitivity in metal loaded cells.

FIG. 7: Doxorubicin uptake by cells upon metal exposure.

Beas-2B cells with and without the 1:128 metal mixture (Table 1) were incubated with varying concentrations of doxorubicin for 1 hour and intracellular fluorescence originating from the doxorubicin was determined. No difference was observed between the metal loaded and metal free cultures.

FIG. 8. Metal loading and chemoresistance in Beas-2B cells.

Beas-2B cells preloaded with metals (“metals then dox” group) have strong chemoresistance whereas concomitant administration (“metals+dox” group) provided only weak chemoresistance. This demonstrates that the protective effect of metals on cancer cells is not a result of an interaction of metals with the drug but a biological effect of metal uptake on the cells.

FIG. 9. Cellular uptake of metals.

MCF7 breast cancer cells (A), Hec-1a endometrial adenocarcinoma cells (B) and Beas-2B bronchial epithelial cells (C) were incubated with metals for 24 hours, washed to remove external metal residues and collected for metal analysis by ICP-MS. It is seen that metal uptake is all cell lines is dose dependent. The results complement and support the dose responsive chemoresistance and p53 unfolding that is observed with increasing doses of metals by demonstrating that the added metals resulted in higher intracellular metal content.

FIG. 10. Metals and p53 in cell lysates.

Beas-2B cell lysates were exposed for 30 min without the metal mix or with metal mix 1:128, 1:256 or 1:512 prior to immunoprecipitation with 240 antibody to detect unfolded p53. Unfolding is detected at all metal mix concentrations, confirming that the unfolding of p53 is a direct effect of the exposure of the protein to the metal mix.

FIG. 11. Metal loading and p53 unfolding in H2170 cells.

H2170 cells were exposed to increasing metal mix combinations for 24 hrs. p53 folding-specific antibodies (240 and 1620) were used to immunoprecipitate p53 and the amount of unfolded and folded p53 was detected on western blot using DO-1 as p53 identifying antibody. Unfolding of p53 was seen with exposure to 1:256 and 1:128 metal mixes.

FIG. 12. Metal loading and p53 mutational status in Hep3B cells.

WT p53, 175 mutant p53, 157 and 158 mutant p53 were transfected in Hep3B cells. Cells were then exposed to 1:512 or 1:64 metals for 8 hrs after which folded and unfolded p53 was immunoprecipitated using 240 or 1620 antibodies. Metal exposure of cells increased p53 unfolding in a dose dependent manner in cells carrying WT p53 and mutants 157 and 158. Surprisingly, the p53 mutants were even more sensitive to low level metals than the WT. The 175 mutant, which is common in many cancers, did not respond visibly to metals as it is already present in the unfolded state in the cells without addition of metals.

FIG. 13. Metals and knockdown p53 in cell lysates.

Beas2B were transfected with siRNA targeting p53. This resulted in a knockdown (kd) of p53 expression as demonstrated on western blot (A). Control (Contr) and kd cells were plated for survival assays, exposed to metals and then treated with doxorubicin as indicated for 72 hrs. Cell survival was monitored and plotted (B) and IC50s were calculated (C). Chemoresistance is attenuated in p53 knockdown as compared to native cells, in accordance with the reduced level of p53 expression. This confirms the findings presented in FIG. 3D that metal induced chemoresistance is mediated by p53.

FIG. 14. Metal exposure and chelation treatment in Beas-2B cells.

Beas-2B cells were pre-treated with metals 1:128 and then exposed to miaDMSA and doxorubicin for 72 hrs after which cell survival was determined (A). Graph B shows IC50. Chemoresistance induced in Beas-2B cells by treatment with a metal mix is completely reversed by chelator miaDMSA.

FIG. 15. Metal exposure and chelation treatment in Beas-2B cells.

Beas-2B cells were pre-treated with metals 1:128 and then exposed to DMSA and doxorubicin for 72 hrs after which cell survival was determined (A). Graph B shows IC50. Chemoresistance induced in Beas-2B cells by treatment with a metal mix is only partially reversed by chelator DMSA.

FIG. 16. Metal exposure and chelation treatment in H2170 cells.

H2170 cells were pre-treated with metals 1:128 and then exposed to DMPS and doxorubicin for 72 hrs after which cell survival was determined (A).

Graph B shows IC50. Chemoresistance induced in H2170 cells by treatment with a metal mix is only partially reversed by chelator DMPS.

FIG. 17. Metal exposure and chelation treatment in BxPC-3 cells.

BxPC3 cells were pre-treated with metals 1:128 and then exposed to 150 μM DMSA or miaDMSA and 5-fluorouracil (5-FU) for 72 hrs, whereafter cell survival was determined (A). Right graph shows viability at 10 μg/ml (B). Chemoresistance to 5-FU is induced in BxPC-3 cells by treatment with a metal mix and reversed by chelators DMSA and miaDMSA.

FIG. 18. Metal exposure and chelation treatment in BxPC-3 cells.

BxPC3 cells were pre-treated with metals 1:128 and then exposed to 150 μM DMSA or miaDMSA and nab-paclitaxel for 72 hrs, whereafter cell survival was determined (A). Graph B shows viability at 60 μg/ml (B). A reduction of resistance was observed with miaDMSA at 60 nM nab-paclitaxel.

FIG. 19. Metal exposure in SW1990 cells.

SW1990 cells were pre-treated with metals 1:128 and then exposed to 150 uM DMSA or miaDMSA and 5-FU for 72 hrs, whereafter cell survival was determined (A). Graph B shows viability at 10 μg/ml. The SW1990 pancreatic adenocarcinoma cell line, which does not have any p53, does not exhibit increased resistance to chemotherapy when exposed to metal. The finding included additional anti-cancer agents (not shown). This observations further confirmed the dependence of metal induced chemoresistance on p53.

FIG. 20. p53 unfolding upon metal exposure and reversal by DMPS.

Beas-2B cells were incubated with metals as indicated in the presence or absence of the chelator DMPS. Folding status of p53 was monitored using the folding antibodies (A). Ratios were determined and indicated in graph B. DMPS prevents unfolding in the presence of metals.

FIG. 21. p53 unfolding upon metal exposure and reversal by DMSA.

Beas-2B cells were incubated in metals as indicated in the presence or absence of the chelator DMSA. Folding status of p53 was monitored using the folding antibodies (A). Ratios were determined and indicated in the graph (B). DMSA prevents unfolding in the presence of metals.

FIG. 22. Unfolding of WT and mutant p53 upon metal exposure and chelation by miaDMSA.

Hep3B cells were transfected with 158 or WT p53 and incubated with metals followed by the chelator miaDMSA as indicated. Folding status of p53 was monitored using the folding antibodies. Unfolding of p53 by metal exposure is prevented or reduced by chelator miaDMSA, in both WT and mutant 158 p53.

FIG. 23. Refolding of p53 in cell lysates upon exposure to chelation treatment.

Beas-2B lysates were incubated with metals for 30 min after which a chelator was added for 1 hr to see if unfolded p53 could be refolded. Subsequently these lysates were subjected to immunoprecipitation with p53 folding antibodies. EDTA and miaDMSA are clearly able to refold some of the p53 in both naïve and loaded cell lysates, indicating that p53 is refolded by chelators rather than being replaced by new protein.

### DETAILED DESCRIPTION OF THE INVENTION

### Definitions

The term ‘a’ or ‘an’, as used herein, for instance in relation to chelating agents or anti-cancer therapeutic agents, includes reference to one or more than one chelating agents. Similarly, it refers to one or more than one anti-cancer therapeutic agents.

The term ‘chelating agent’, interchangeably used with the term ‘chelator’, as used herein, includes reference to an agent that reacts with metal ions to form a metal-chelating agent complex, or chelate. This type of complexing generally involves the formation or presence of two or more separate coordinate bonds between a polydentate ligand and a single central atom. Such ligands are also referred to as chelants, chelators, chelating agents, or sequestering agents. The skilled person is well aware of the vast array of chelating agents available, including but not limited to Acetylacetone, Alizarin, Alizarin Red S, Amidoxime, Amidoxime group, Aminoethylethanolamine, Aminomethylphosphonic acid, Aminopolycarboxylic acid, ATMP, BAPTA, Bathocuproine, BDTH2, Benzotriazole, Bipyridine, 2,2′-Bipyridine, 2,2′-Bipyrimidine, Bis(dicyclohexylphosphino) ethane, 1,2-Bis(dimethylarsino)benzene, 1,2-Bis(dimethylphosphino) ethane, 1,4-Bis(diphenylphosphino) butane, 1,2-Bis(diphenylphosphino) ethane, Calixarene, Carcerand, Catechol, Cavitand, Citrate, Citric acid, Clathrochelate, Corrole, 2,2,2-Cryptand, Cyclam, Cyclen, Cyclodextrin, Deferasirox, Deferiprone, Deferoxamine, Denticity, Dexrazoxane, Diacetyl monoxime, Trans-1,2-Diaminocyclohexane, 1,2-Diaminopropane, 1,5-Diaza-3,7-diphosphacyclooctanes, 1,4-Diazacycloheptane, 1,5-Diazacyclooctane, Dibenzoylmethane, Diethylenetriamine, Diglyme, 2,3-Dihydroxybenzoic acid, Dimercaprol, 2,3-Dimercapto-1-propanesulfonic acid, Dimercaptosuccinic acid, 1,1-Dimethylethylenediamine, 1,2-Dimethylethylenediamine, Dimethylglyoxime, DIOP, Diphenylethylenediamine, 1,5-Dithiacyclooctane, Domoic acid, DOTA, DOTA-TATE, DTPMP, EDDHA, EDDS, EDTA, EDTMP, EGTA, 1,2-Ethanedithiol, Ethylenediamine, Ethylenediaminediacetic acid, Ethylenediaminetetraacetic acid, Etidronic acid, Fluo-4, Fura-2, Gallic acid, Gluconic acid, Glutamic acid, Glyoxal-bis(mesitylimine), Glyphosate, Hexafluoroacetylacetone, Homocitric acid, Iminodiacetic acid, Indo-1, Isosaccharinic acid, Kainic acid, Malic acid, Metal acetylacetonates, Metal dithiolene complex, Metallacrown, Nickel bis(stilbenedithiolate), Nitrilotriacetic acid, Oxalic acid, Oxime, Pendetide, Penicillamine, Pentetic acid, Phanephos, Phenanthroline, O-Phenylenediamine, Phosphonate, Phthalocyanine, Phytochelatin, Picolinic acid, Polyaspartic acid, Porphine, Porphyrin, 3-Pyridylnicotinamide, 4-Pyridylnicotinamide, Pyrogallol, Salicylic acid, Sarcophagine, Sodium citrate, Sodium diethyldithiocarbamate, Sodium polyaspartate, Terpyridine, Tetramethylethylenediamine, Tetraphenylporphyrin, Tetrasodium EDTA, Thenoyltrifluoroacetone, Thioglycolic acid, TPEN, 1,4,7-Triazacyclononane, Tributyl phosphate, Tridentate, Triethylenetetramine, 1,1,1-Trifluoroacetylacetone, 1,4,7-Trimethyl-1,4,7-triazacyclononane, Triphos, Trisodium citrate, 1,4,7-Trithiacyclononane and TTFA. Chelating agents may be lipophilic, such as dimercaprol (BAL), deferasirox (marketed as Exjade™, Desirox™, Defrijet™, Desifer™, Rasiroxpine™ and Jadenu™), N,N′-bis(2-mercaptoethyl) isophthalamide (also referred to as BDTH2, BDET and BDETH2; trade names B9™, MetX™ and OSR #1™), prussian blue (Radiogardase™), α-lipoic acid, mono-alkylated DMSA (e.g. monoisoamylDMSA), oximes (e.g. dimethylglyoxime, salicylaldoxime), diethyldithiocarbamate (DDC) and derivatives thereof (e.g. N(methoxybenzyl)-Dglucamine dithiocarbamate) and dexrazoxane; or hydrophilic, such as dimercaptosuccinic acid (DMSA), dimercaptopropanoic acid (DMPA) or derivatives thereof, ethylenediaminetetraacetic acid (EDTA), diethylenetriaminepentaacetic acid (DTPA), N-acetylcysteine (NAC), malic acid, succinic acid, citric acid, tartaric acid, deferoxamine, penicillamine, nitrilotriacetate (NTA), histidine, 2,3-dimercapto-1-propanesulfonic acid (DMPS). When reference is made to a specific chelator, such as DMSA, included within said definition are derivatives thereof. As an example, monoisoamylDMSA (miaDMSA) is a derivative of DMSA. The term ‘chelator’ includes reference to the metal complexes of chelators such as, for EDTA, Ca-EDTA, sodium calcium EDTA and Zn-EDTA, and for DTPA for instance Zn-DTPA, Ca-DTPA, etc.

The term “broad-spectrum chelating agent”, as used herein, includes reference to a chelating agent that is capable of chelating different metals such as at least 2, 3, 4, 5 or at least 6 different metals.

Preferably, the chelating agent as disclosed herein is a broad-spectrum chelating agent. Examples of broad-spectrum chelating agents are EDTA, DMSA, DMPS, DTPA, BAL, etc. Preferably, the chelating agent as disclosed herein is miaDMSA.

The term ‘tumor’, as used herein, includes reference to an abnormal growth of tissue that may be benign, pre-cancerous, malignant, or metastatic. The tumor is preferably malignant, i.e., a cancer. Examples of cancers include, but are not limited to, breast cancer, prostate cancer, ovarian cancer, cervical cancer, skin cancer, pancreatic cancer, spleen cancer, colorectal cancer, renal cancer, liver cancer, brain cancer, lymphoma, leukemia such as acute myeloid leukemia (AML), Hodgkin lymphoma, non-Hodgkin lymphoma, lymph nodes cancer, bone marrow cancer, lung cancer, stomach cancer, eye cancer and the like. The term ‘liquid tumor’, as used herein, includes reference to a form of cancer wherein the cancer cells are primarily situated in body fluids, such as blood, bone marrow and lymph. The term ‘solid tumor’, as used herein, includes reference to a form of cancer wherein the cancer cells are primarily situated in a solid tissue, such as tissue of the lung, heart, brain, spleen, pancreas, liver, breast, prostate, bowel, stomach, bone, skin and cartilage.

The terms ‘treatment’ and ‘treating’, as used herein, include reference to the application of a form of therapy to a subject, with the object of e.g. curing the patient from a disease, halting or slowing down the development of a disease, prolonging the life of a subject, or relieving pain in a subject suffering from a disease or injury. For example, a treatment may include chemotherapy, immunotherapy, radiotherapy, performance of surgery, and any combination thereof. Prophylactic treatment, therapy with the aim of preventing induction or onset of a disease, is also to be understood to be part of the term ‘treatment’.

The term ‘subject’, as used herein, includes reference to a recipient of a chelating agent as described herein, i.e., a subject that is suffering, or suspected of suffering, from cancer. Preferably, the subject is a mammal, more preferably a human. The terms “patient” and “subject” can be used interchangeably herein. The subject is preferably a human, more preferably a human having a cancer. The subject can be older or younger than 50 years old, preferably older than 50 years old.

The term ‘resistance’, as used herein, includes reference to the ability of cancer cells to at least partially withstand one or more anti-cancer therapies, particularly one or more chemotherapies. In other words, resistance refers inter alia to a reduced efficacy of an anti-cancer therapeutic agent to treat a subject having a cancer with elevated metal levels as compared to the efficacy achieved with said anti-cancer therapeutic agent in the treatment of a subject having a cancer without said elevated metal levels. When the anti-cancer therapeutic agent is a chemotherapeutic agent, the resistance is referred to as chemoresistance. When reference to a resistance of a cancer is made, it preferably refers to a resistance of cancer cells of said cancer to said anti-cancer therapeutic agent. Preferably, the resistance is a p53-dependent resistance or a metal-induced resistance such as a p53-dependent resistance that is metal-induced.

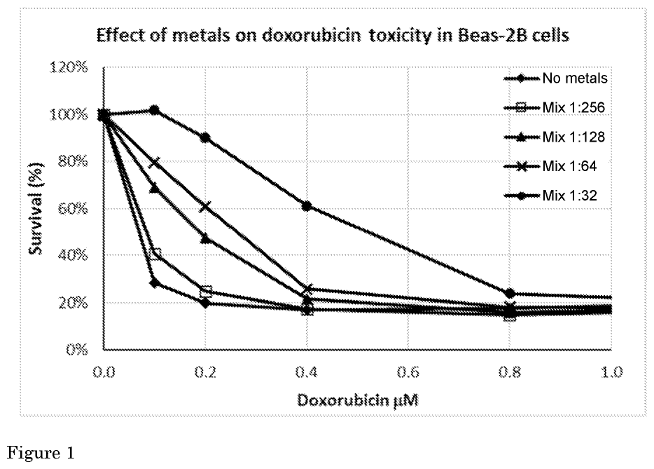

The term ‘metal-induced resistance’, as used herein, includes reference to resistance that is induced as a result of exposure to one or more metals, preferably multiple metals (i.e., a multi-metal exposure), more preferably elevated levels of said multiple metals, such as at least 2, 3, 4, 5, 6, 7, 8, 9, 10, 11, 12, 13, 14, 15, 16, 17, 18, 19, 20, 21, 22 or at least 23 metals selected from the list formed by arsenic (As), aluminum (Al), antimony (Sb), Barium (Ba), boron (B), cadmium (Cd), Cerium (Ce), Chromium (Cr), lead (Pb), mercury (Hg), neodymium (Nd), manganese (Mn), Nickel (Ni), tin (Sn), titanium (Ti), uranium (U), vanadium (V), copper (Cu), iron (Fe), gold (Au), silver (Ag), palladium (Pd) and platinum (Pt). The presence of elevated metal levels, as disclosed herein, can e.g. be determined using the multi-metal scoring system disclosed in FIG. 1 of Ohanian et al., Am J Hematol.2020; 95:422-434, which includes relevant metal level cut-off values. The contents of Ohanian et al., Am J Hematol.2020; 95:422-434 are incorporated herein by reference. Preferably, metal-induced resistance is caused by metal-induced aberrant folding of a tumor suppressor protein such as p53 resulting in a resistance to an anti-cancer therapeutic agent such as a chemotherapeutic agent, e.g. doxorubicin. Aberrant folding can be a misfolding and/or unfolding of a tumor suppressor protein such as p53. The skilled person can assess p53 folding status in cancer cells e.g. by the p53 folding status assay described in the Examples.

As an alternative to using the term “resistance” such as in “chemoresistance” and counteracting it in the medical use/methods of the invention, one could instead refer to restoring sensitivity to therapy, such as restoring chemosensitivity. Hence, the phrase “counteracting a (chemo) resistance can be used interchangeably with the phrase “restoring (chemo) sensitivity”.

The term ‘restoring’, as used herein in relation to restoring chemosensitivity, includes reference to at least partial, such as complete, restoration of chemosensitivity of cancer to an anti-cancer therapeutic agent it was previously at least partially insensitive to.

The term ‘anti-cancer therapeutic agent’, as used herein, includes reference to substances, drugs, therapeutics and/or compositions that are applied to a subject having cancer with the aim of treating said subject. An anti-cancer therapeutic agent may be used as part of for example chemotherapy, immunotherapy, stem cell therapy, hormone therapy, radiation therapy and/or surgery. Most anti-cancer therapeutic agents have an anti-cancer effect for at least one type of cancer in at least one individual. The anti-cancer therapeutic agent as disclosed herein can be a chemotherapeutic agent, a targeted anti-cancer therapeutic agent such as an immunotherapeutic agent (e.g. an immune checkpoint inhibitor), etc.

The term ‘anti-cancer effect’, as used herein, includes reference to the effect that an anti-cancer therapeutic agent has on a cancer. An anti-cancer effect may for example be a cytotoxic effect and/or a cytostatic effect. The term ‘cytotoxic effect’, as used herein, includes reference to an anti-cancer effect wherein the therapy leads to damage and subsequent death of a cancer cell. In general, chemotherapeutic agents have a cytotoxic effect. The term ‘cytostatic effect’, as used herein, includes reference to an anti-cancer effect wherein the therapy leads to the inhibition of cell growth and/or multiplication.

The term ‘sensitizing’, as used herein, includes reference to the application of a chelating agent, preferably a broad-spectrum chelating agent, that, as part of anti-cancer therapy, makes the cancer more susceptible to an anti-cancer therapeutic agent, preferably an anti-cancer therapeutic agent to which the cancer has a reversible resistance. This does not exclude the possibility that the chelating agent also in itself has an anti-cancer effect. The term ‘chemosensitizing’, as used herein, includes reference to sensitizing with the aim of making a cancer more susceptible to chemotherapy, wherein chemotherapy is applied in combination with the chelation therapy.

The term ‘reversal’ or ‘reversing’, as used herein, includes reference to partial or complete reversal of a resistance that a cancer (preferably cancer cells of said cancer) has towards an anti-cancer therapeutic agent.

The term ‘combination’ or ‘combination therapy’, as used herein, includes reference to using a chelating agent as disclosed herein and an anti-cancer therapeutic agent as disclosed herein in the same medical treatment. The chelating agent and the anti-cancer therapeutic agent as disclosed herein can be administered together at the same time (such as in the form of a single pharmaceutical composition), separately of each other at the same time (for instance in the form of separate pharmaceutical composition) or separately of each other staggered in time. Simultaneous, separate and sequential administration of a chelating agent and an anti-cancer therapeutic agent as disclosed herein in the same treatment schedule are expressly envisaged. As an example, the time between administration of said chelating agent and said anti-cancer therapeutic agent can be at least one minute, at least fifteen minutes, at least sixty minutes, at least four hours, at least one day, at least one week or at least one month or at least one year, or anywhere in between such as between one minute and one year. Preferably, the chelating agent is administered prior to administration of said anti-cancer therapeutic agent. Alternatively, the anti-cancer therapeutic agent is administered together with, or after, administration of said chelating agent.

The term ‘pharmaceutical combination’, as used herein, includes reference to e.g. a kit of parts containing multiple containers that hold the different active ingredients.

The term ‘therapeutically effective amount’, as used herein, means that the amount of active ingredients administered is of sufficient quantity to achieve the intended purpose, such as, in this case, for the chelating agent, to reverse chemoresistance or to restore chemosensitivity.

The term ‘potentiating’, as used herein, includes reference to the application of a chelating agent that, as part of anti-cancer therapy, makes the cancer more susceptible to an anti-cancer therapeutic agent to thereby enhance the anti-cancer effects of said anti-cancer therapeutic agent.

The term ‘metal’, as used herein, includes reference to a chemical element that may occur in the body of a subject. Preferably, said metal is present in the body in ionic form. Metalloids should also be understood to fall under the definition of ‘metal’ in the context of the invention. Elements that are commonly referred to as metals or metalloids, are lithium, beryllium, sodium, magnesium, aluminum, potassium, calcium, scandium, titanium, vanadium, chromium, manganese, iron, cobalt, nickel, copper, zinc, gallium, rubidium, strontium, yttrium, zirconium, niobium, molybdenum, technetium, ruthenium, rhodium, palladium, silver, cadmium, indium, tin, cesium, barium, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold, mercury, thallium, lead, bismuth, polonium, francium, radium, actinium, thorium, protactinium, uranium, neptunium, plutonium, americium, curium, berkelium, californium, einsteinium, fermium, mendelevium, nobelium, lawrencium, rutherfordium, dubnium, seaborgium, bohrium and hassium. Elements that are commonly referred to as metalloids, and thus are also referred to by the term ‘metal’ in this document, include boron, silicon, germanium, arsenic, antimony, and tellurium.

The term ‘tumor suppressor (protein) function’, as used herein, includes reference to the capacity of a tumor suppressor protein to negatively regulate the cell cycle and/or promote apoptosis. Hence, when reference to tumor suppressor (protein) function is made, it preferably refers to negative regulation of the cell cycle and/or promotion of apoptosis. Preferably, the tumor suppressor protein function refers to the wild-type tumor suppressor protein function, not to aberrant tumor suppressor protein function that is the result of gain of function or loss of function mutation. Non-limiting examples of tumor suppressor proteins are retinoblastoma protein, p16, p53, FAS, NOTCH receptors, VHL, APC, MSH2, BRCA2, neurofibromin, and PTCH1. Preferably, the tumor suppressor protein is p53.

The term ‘impaired’, as used herein in relation to tumor suppressor (protein) function, includes reference to a biologically relevant reduction in, or inactivation of, tumor suppressor (protein) function that is preferably the result of aberrant tumor suppressor protein folding caused by elevated levels of metals. Preferably, the aberrant tumor suppressor protein folding is a metal-induced aberrant tumor suppressor protein folding.

The term ‘inactivation’, as used herein, includes reference to an at least partial reduction or complete abolishment of tumor suppression function, e.g. p53 tumor suppressor function. Inside a cell, a population of the same protein is present. If for said population the tumor suppressor activity is substantially reduced or completely abolished, it may be considered inactivated.

The term ‘aberrant’, as used herein in relation to tumor suppressor protein folding, includes reference to incorrect (e.g. non-wild-type) folding of a tumor suppressor protein. Aberrant folding preferably is to the extent that the protein cannot exert its normal (preferably wild-type) protein function because it is not in its normal (preferably wild-type) conformation. The aberrant folding can for instance be an aberrant protein misfolding, an aberrant protein unfolding or an aberrant protein aggregation. Preferably, the aberrant folding is caused by elevated levels of metals inside cancer cells. Preferably, the aberrant tumor suppressor protein folding is an (elevated) metal-induced aberrant tumor suppressor protein folding.

In preferred embodiments, the cancer has a wildtype p53 or a mutated p53. For example a mutated p53 having aberrant protein folding when subjected to elevated metal levels. For example the mutated p53 is susceptible to re-folding back to the native protein conformation of wild-type p53. When cancer cells of said cancer express a mutated p53, said p53 comprises one or more mutations as compared to wildtype p53 which result in aberrant protein folding when subjected to elevated levels of one or more metals but fold normally when not subjected to elevated levels of said one or more metals. Mutated p53 protein is for example as sensitive as wildtype p53 protein to metals. For example mutated p53 protein is more sensitive to metals than wildtype p53 protein, such that mutated p53 protein will unfold at lower metal levels than wildtype p53 protein. As a result of mutated p53 protein being more sensitive to metals than wildtype p53 protein, administration of chelating agents in subjects and/or cancers having mutated p53 proteins will already facilitate re-folding and re-activation/restoration of tumor suppressor function and thus, reversal of chemoresistance under moderate (i.e., normal or near normal) metal levels. The skilled person can test for mutations with these functions by expressing the mutated p53 protein in vitro, subjecting it to a certain metal load, and assess p53 folding status, the latter e.g. as described in the Examples section. The skilled person will understand that a cell contains multiple copies of a protein, and that folding status of the protein population in a cell is not absolute in the sense that all proteins are either aberrantly folded or normally folded.

The term ‘unfolding’, as used herein, includes reference to the partial or full abolishment of a protein's tertiary and/or quaternary structure, also called its conformation. The term ‘unfolding’ should both be viewed as unfolding as part of the protein folding process, but also as unfolding (partial or full unfolding) of the protein's normal (wild-type) conformation. Unfolding may for example occur as a result of the presence of elevated metal levels. An unfolded protein has a different tertiary and/or quaternary structure than the same protein in its native (wild-type) conformation. Aberrant folding may lead to loss of function and/or gain of function.

The term ‘misfolding’, as used herein, includes reference to folding of a protein that leads to a non-native folding state, wherein the tertiary and/or quaternary structure of the protein differs from that of the native (wild-type) folded protein. Misfolding can both be the result of an intermediate yet incomplete step of the native folding process, or a change in normal folding as a result of an environmental change. This may for example occur as a result of the presence of elevated metal levels of one or more metals and/or as a result of one or more mutations in a tumor suppressor gene such as p53. Misfolding may lead to loss of function and/or gain of function.

The term ‘aggregation’, as used herein, includes reference to the accumulation by binding of two or more unfolded and/or misfolded proteins. The term aggregation refers for example to pathological protein aggregation. Aggregation may for example occur as a result of the presence of metal levels and/or as a result of one or more mutations such as mutation in a tumor suppressor protein gene. Aggregation may lead to loss of function and/or gain of function.

The term ‘chemotherapeutic agent’, as used herein, includes reference to an anti-cancer drug that is used as part of a chemotherapy, which is a type of cancer treatment. A chemotherapeutic agent may have a cytotoxic effect and/or a cytostatic effect, and it may be used to cure a subject from cancer, to reduce symptoms in a subject, or to prolong the life of a subject. Non-limiting examples of a chemotherapeutic agent include Cyclophosphamide, Mechlorethamine, Chlorambucil, Melphalan, Dacarbazine, Nitrosoureas, Temozolomide, Daunorubicin, Doxorubicin, Epirubicin, Idarubicin, Mitoxantrone, Valrubicin, Cabazitaxel, Larotaxel, Ortataxel, Tesetaxel, Paclitaxel, Docetaxel, Abraxane, Taxotere, Epothilone A, Epothilone B, Epothilone C, Epothilone D, Epothilone E, Epothilone F, Vorinostat, Romidepsin, Irinotecan, Topotecan, Etoposide, Teniposide, Tafluposide, Bortezomib, Erlotinib, Gefitinib, Imatinib, Vemurafenib, Vismodegib, Azacitidine, Azathioprine, Capecitabine, Cytarabine, Doxifluridine, Fluorouracil, Gemcitabine, Hydroxyurea, Mercaptopurine, Methotrexate, Tioguanine, Bleomycin, Actinomycin, Carboplatin, Cisplatin, Oxaliplatin, Nedaplatin, Triplatin Tetranitrate, Phenanthriplatin, Picoplatin, Satraplatin, Tretinoin, Alitretinoin, Bexarotene, Vinblastine, Vincristine, Vindesine, Vinflunine, Vinorelbine, Aminopterin, Pemetrexed, Pralatrexate, Raltitrexed, Pentostatin, Cladribine, Clofarabine, Fludarabine, Nelarabine, Carmofur, Floxuridine, Tegafur, Cytarabine, Gemcitabine, Decitabine, Hydroxycarbamide, Belotecan, Camptothecin, Cositecan, Etirinotecan pegol, Exatecan, Gimatecan, Lurtotecan, Rubitecan, Silatecan, Aclarubicin, Amrubicin, Pirarubicin, Valrubicin, Zorubicin, Mitoxantrone, Losoxantrone, Pixantrone, Bendamustine, Chlormethine, Ifosfamide, Trofosfamide, Prednimustine, Uramustine, Carmustine, Fotemustine, Lomustine Semustine, Nimustine, Ranimustine, Streptozocin, Mannosulfan, Treosulfan, Carboquone, Thiotepa, Triaziquone, Triethylenemelamine, Altretamine, Procarbazine, Mitobronitol, Pipobroman, Dacarbazine, Temozolomide, Dactinomycin, Bleomycin, Mitomycins, Plicamycin, Aminolevulinic acid, Efaproxiral, Methyl aminolevulinate, Padeliporfin, Porfimer sodium, Talaporfin, Temoporfin, Verteporfin, Tipifarnib, Abemaciclib, Alvocidib, Palbociclib, Ribociclib, Seliciclib, Bortezomib, Carfilzomib, Oprozomib, Ixazomib, Anagrelide, Tiazofurin, Masoprocol, Niraparib, Olaparib, Rucaparib, Belinostat, Entinostat, Panobinostat, Romidepsin, Vorinostat, Pi3K, Alpelisib, Copanlisib, Duvelisib, Idelalisib, Umbralisib, Atrasentan, Bexarotene, Testolactone, Amsacrine, Arsenic trioxide, Asparaginase, Pegaspargase, Belzutifan, Celecoxib, Demecolcine, Elesclomol, Elsamitrucin, Eribulin, Estramustine phosphate, Etoglucid, Lonidamine, Lucanthone, Mitoguazone, Mitotane, Oblimersen, Omacetaxine mepesuccinate, Trabectedin, Alitretinoin, Bexarotene, Tretinoin, Veliparib and Venetoclax. Combinations of chemotherapeutic agents may be used for the treatment of cancer. Combinations may be established or improvised by the treating physician. Established combinations are known as regimens, wherein in some cases also dose and administration interval are included.

The chemotherapeutic agent as disclosed herein can be an (i) alkylating agent (such as Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa or Trabectedin), (ii) an nitrosoureas (such as Carmustine, Lomustine or Streptozocin), (iii) an antimetabolite (Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine or Trifluridine/tipiracil combination, (iv) an anthracycline (such as Daunorubicin, Doxorubicin (Adriamycin), Doxorubicin liposomal, Epirubicin, Idarubicin or Valrubicin), (v) an topoisomerase I or II inhibitor (such as Irinotecan, Irinotecan liposomal, Topotecan, Etoposide (VP-16), Mitoxantrone or Teniposide), (vi) a taxane (Cabazitaxel, Docetaxel, Nab-paclitaxel or Paclitaxel), (vii) a vinca alkaloid (such as Vinblastine, Vincristine, Vincristine liposomal or Vinorelbine), (viii) a corticosteroid (such as Prednisone, Methylprednisolone or Dexamethasone), or (ix) All-trans-retinoic acid, Arsenic trioxide, Asparaginase, Eribulin, Hydroxyurea, Ixabepilone, Mitotane, Omacetaxine, Pegaspargase, Procarbazine, Romidepsin or Vorinostat.

The term ‘anthracycline’, as used herein, includes reference to a member of the anthracyclines, a group of chemotherapeutic agents comprising doxorubicin, daunorubicin, epirubicin and idarubicin or their derivatives. Those chemotherapeutic agents are naturally produced by the bacterium Streptomyces peucetius. The term ‘doxorubicin’, as used herein, includes reference to a chemotherapeutic agent belonging to the group of anthracyclines. It is sold under trade names including Adriamycin, Doxil, Caelyx and Myocet. It is used for the treatment of cancers, including breast cancer, bladder cancer, Kaposi's sarcoma, lymphoma, and acute lymphocytic leukemia. Doxorubicin is part of several chemotherapeutic regimens, including AC, TAC, ABVD and FAC.

The term ‘administration’, as used herein, includes reference to the application of a substance or composition to a subject. Main routes of administration are parenteral administration, enteral or gastrointestinal administration and topical administration. The term ‘parenteral’, as used herein, includes reference to any form of administration that is not via the application onto the skin or via the gastrointestinal tract. Non-limiting examples of parenteral administration include epidural, intracerebral, intracerebroventricular, epicutaneous, sublingual, extra-amniotic, nasal, intra-arterial, intra-articular, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, rectal or intratumoral administration. The term ‘intravenous’, as used herein, includes reference to a parenteral route of administration wherein a substance or composition is injected into the vein of a subject, for example using a hollow needle. The substance or composition that is administered intravenously will directly reach the blood stream of the subject. The term ‘intratumoral’, as used herein, includes reference to administration of a substance or composition directly into a tumor, for example using a hollow needle. The tumor wherein intratumoral administration takes place may be treated prior to administration, for example in order to improve visibility of the tumor. Intratumoral administration may for example be used for the administration of anti-cancer therapeutic agents.

### Chelators

The present invention relates to a chelating agent for use in a method of treating cancer in a subject, wherein said cancer has a resistance to an anti-cancer therapeutic agents. Preferably, said chelating agent is for use in combination with an anti-cancer therapeutic agent as disclosed herein. For example, said chelating agent is for use in combination with the anti-cancer therapeutic agent to which the cancer was resistant and/or with a different anti-cancer therapeutic agent. It has been found that the use of a chelating agent in a subject that is resistant to one or more anti-cancer agents is beneficial from a treatment perspective.

Without being bound by theory, the chelating agent binds metals in the body. Metals may induce improper folding of proteins. Once metals are chelated, proteins are less likely to improperly fold, thereby restoring the function of said proteins in the body. A protein that commonly misfolds in the presence of metals and as a result loses its wild-type tumor suppressor function and conveys chemoresistance, is p53. The inventors established that chelation results in the restoration of wild-type p53 function, and in the alleviation of chemoresistance.

Preferably, the chelating agent binds to metals such as a lead, chromium, arsenic, mercury, cadmium, aluminum, antimony, barium, bismuth, copper, gold, iron, lithium, manganese, nickel, vanadium, platinum, silver, thallium, tin and/or titanium.

In some embodiments, the chelating agent is selected from the group comprising Acetylacetone, Alizarin, Alizarin Red S, Amidoxime, Amidoxime group, Aminoethylethanolamine, Aminomethylphosphonic acid, Aminopolycarboxylic acid, ATMP, BAPTA, Bathocuproine, BDTH2, Benzotriazole, Bipyridine, 2,2′-Bipyridine, 2,2′-Bipyrimidine, Bis(dicyclohexylphosphino) ethane, 1,2-Bis(dimethylarsino)benzene, 1,2-Bis(dimethylphosphino) ethane, 1,4-Bis(diphenylphosphino) butane, 1,2-Bis(diphenylphosphino) ethane, Calixarene, Carcerand, Catechol, Cavitand, Citrate, Citric acid, Clathrochelate, Corrole, 2,2,2-Cryptand, Cyclam, Cyclen, Cyclodextrin, Deferasirox, Deferiprone, Deferoxamine, Denticity, Dexrazoxane, Diacetyl monoxime, Trans-1,2-Diaminocyclohexane, 1,2-Diaminopropane, 1,5-Diaza-3,7-diphosphacyclooctanes, 1,4-Diazacycloheptane, 1,5-Diazacyclooctane, Dibenzoylmethane, Diethylenetriamine, Diglyme, 2,3-Dihydroxybenzoic acid, Dimercaprol (BAL), 2,3-Dimercapto-1-propanesulfonic acid (DMPS), Dimercaptosuccinic acid (DMSA), miaDMSA, 1,1-Dimethylethylenediamine, 1,2-Dimethylethylenediamine, Dimethylglyoxime, DIOP, Diphenylethylenediamine, 1,5-Dithiacyclooctane, Domoic acid, DOTA, DOTA-TATE, DTPMP, EDDHA, EDDS, ethylenediaminetetraacetic acid (EDTA), EDTMP, EGTA, 1,2-Ethanedithiol, Ethylenediamine, Ethylenediaminediacetic acid, Ethylenediaminetetraacetic acid, Etidronic acid, Fluo-4, Fura-2, Gallic acid, Gluconic acid, Glutamic acid, Glyoxal-bis(mesitylimine), Glyphosate, Hexafluoroacetylacetone, Homocitric acid, Iminodiacetic acid, Indo-1, Isosaccharinic acid, Kainic acid, Malic acid, Metal acetylacetonates, Metal dithiolene complex, Metallacrown, Nickel bis(stilbenedithiolate), Nitrilotriacetic acid, Oxalic acid, Oxime, Pendetide, Penicillamine, Pentetic acid, Phanephos, Phenanthroline, O-Phenylenediamine, Phosphonate, Phthalocyanine, Phytochelatin, Picolinic acid, Polyaspartic acid, Porphine, Porphyrin, 3-Pyridylnicotinamide, 4-Pyridylnicotinamide, Pyrogallol, Salicylic acid, Sarcophagine, Sodium citrate, Sodium diethyldithiocarbamate, Sodium polyaspartate, Terpyridine, Tetramethylethylenediamine, Tetraphenylporphyrin, Tetrasodium EDTA, Thenoyltrifluoroacetone, Thioglycolic acid, TPEN, 1,4,7-Triazacyclononane, Tributyl phosphate, Tridentate, Triethylenetetramine, 1,1,1-Trifluoroacetylacetone, 1,4,7-Trimethyl-1,4,7-triazacyclononane, Triphos, Trisodium citrate, 1,4,7-Trithiacyclononane, TTFA, N,N′-bis(2-mercaptoethyl) isophthalamide (also referred to as BDTH2, BDET and BDETH2), prussian blue, α-lipoic acid, mono-alkylated DMSA, diethyldithiocarbamate (DDC), dimercaptopropanoic acid (DMPA) or derivatives thereof, diethylenetriaminepentaacetic acid (DTPA), N-acetylcysteine (NAC), succinic acid, tartaric acid, nitrilotriacetate (NTA), and histidine, or combinations thereof.

In a medical use of the invention, at least two, at least three, at least four or at least five chelating agents can be employed, e.g. partially or fully selected from the above-listed chelating agents. Preferably, at least one chelating agent is selected from the group comprising DMPS, EDTA, DTPA, dimercaprol, DMSA, and derivatives thereof, such as miaDMSA, or combinations thereof. More preferably, said at least one chelating agent is a combination of DMPS and EDTA; of DMPS and DTPA; of DMPS and dimercaprol; of DMPS and DMSA, preferably miaDMSA; of EDTA and DTPA; of EDTA and dimercaprol; of EDTA and DMSA, preferably miaDMSA; of DTPA and dimercaprol; of DTPA and DMSA, preferably miaDMSA; of dimercaprol and DMSA, preferably miaDMSA. Even more preferably, one or more chelating agent is selected from the group comprising DMSA, DMPS, EDTA, and derivatives thereof, such as miaDMSA. In a preferred embodiment, the chelating agent is DMPS, a combination of at least DMPS and EDTA, or a combination of at least DMSA, preferably miaDMSA, and EDTA.

In a medical use of the invention, the chelating agent can for instance be used in a dose range of 1-100 mg/kg, preferably 2-50 mg/kg.

Preferably, the chelating agent as disclosed herein has binding properties which allow for chelation of at least one metal selected from the group comprising vanadium, chromium, iron, copper, lead, arsenic, mercury and cadmium. More preferably, said chelating agent is able to chelate at least one metal selected from the group comprising iron, copper, lead, mercury, cadmium and vanadium. Even more preferably, said chelating agent is able to chelate a combination of metals selected from the group comprising iron, copper, lead, mercury and cadmium.

The chelating agent as disclosed herein can be used in a method of sensitizing a cancer of a subject to an anti-cancer therapeutic agent. Preferably, said sensitizing comprises sensitizing a cancer of a subject to an anti-cancer therapeutic agents, wherein said cancer has a resistance to said anti-cancer therapeutic agents. More preferably, said sensitizing comprises chemosensitizing a cancer of a subject to a chemotherapeutic agent, wherein said cancer has a resistance to said chemotherapeutic agents. Even more preferably, said chemosensitizing comprises sensitizing a cancer of a subject to a chemotherapeutic agent, wherein said cancer has a p53-dependent resistance to said chemotherapeutic agent. Still more preferably, said p53-dependent resistance to said one or more chemotherapeutic agents is caused by aberrant p53 folding.

A chelating agent as disclosed herein can be for use in a method of potentiating an anti-cancer therapeutic agent as described herein. Preferably, the anti-cancer effect of said anti-cancer therapeutic agent is potentiated. As a result, the anti-cancer effect of said anti-cancer therapeutic agent is enhanced. Preferably, said enhancement of the anti-cancer effect is more than 10%; more preferably more than 25%; even more preferably more than 50%; still more preferably more than 100%; most preferably more than 200% as compared to treatment with said anti-cancer therapeutic agent alone.

In a medical use of the invention, the chelating agent as disclosed herein can be administered to said subject in combination with a further agent, such as a reducing agent. Examples of reducing agents are an antioxidant and a vitamin. Alternatively, said chelating agent can be administered in combination with an essential mineral. Said further agent is preferably administered in combination with a chelating agent as disclosed herein and/or with an anti-cancer therapeutic agent as disclosed herein. Suitable antioxidant vitamins include ascorbic acid (vitamin C) and α-tocopherol (vitamin E). Other antioxidant compounds include glutathione, lipoic acid, uric acid, carotenoids (e.g., beta-carotene, lycopene), flavonoids (e.g. quercetin), retinol, ubiquinol (coenzyme Q), taurine, N-acetylcysteine (NAC), and amifostine. Essential minerals are those minerals which are necessary for proper functioning of the body. They are sometimes classified into i) macrominerals such as, chloride, calcium, sodium, phosphorus, potassium, magnesium, and sulfur; and microminerals (trace minerals) such as zinc, selenium, magnesium, calcium, and rubidium; a subset is zinc, selenium, magnesium, and calcium. One or more of these antioxidants and/or one or more of these essential minerals can be administered in combination with said chelating agent.

In a medical use of the invention, at least one, more preferably two, more preferably three, and even more preferably all of the following vitamins and minerals are administered to a subject: zinc, selenium, magnesium, and/or vitamin C; preferably, these compounds are administered to the subject in combination with said chelating agent and/or said anti-cancer therapeutic agent as disclosed herein. Examples of ranges of dosages and forms of zinc, selenium, magnesium, and vitamin C that can be administered to a subject or patient can include but are not limited to the following: zinc (e.g., elemental zinc, zinc sulfate, zinc citrate, or zinc glycenate at 5-75 mg, such as 50 mg), vitamin C (e.g., 1000 mg to 50 grams, such as orally or intravenously), magnesium citrate (e.g., 100 mg to 3 g, such as orally or intravenously, such as 3 g magnesium sulfate IV), and selenium (e.g., L-Selenomethionine or equivalent, such as 100-200 mcg orally daily). In some embodiments, one or more, or all, of the following vitamins and minerals are administered to a subject: magnesium, selenium, rubidium, zinc, and/or vitamin C.

EDTA can be used as the chelating agent. Said EDTA may also be provided in the form of calcium-EDTA (Ca-EDTA), zinc-EDTA (Zn-EDTA), sodium-EDTA (Na-EDTA), potassium EDTA or sodium calcium EDTA. In embodiments, said EDTA is in a dose, e.g. single unit dose, of 40-12000 mg. In some embodiments, DMPS and/or DMSA, preferably miaDMSA, is used as the chelating agent. DMSA can e.g. be in the form of Zn-DMSA. DMPS can e.g. be in the form of Zn-DMPS. As a non-limiting example, DMPS and/or DMSA, preferably miaDMSA, may be administered at a concentration of 10-30 mg/kg/day.

Preferably, said DMPS and/or DMSA, preferably miaDMSA, is in a dose, e.g. single unit dose, of 40-12000 mg, for instance 40-6000 mg, 100-5000 mg, 200-4000 mg or 400-3600 mg.

### Cancer

A chelating agent as disclosed herein is for use in the treatment of cancer. A cancer as described herein may be any cancer. Preferably, said cancer is a cancer wherein p53 tumor suppressor function is impaired such as at least partially, or completely, inactivated. More preferably, said inactivation of said tumor suppressor function of p53 is not solely mediated by one or more mutations in the p53 gene. As an example, inactivation of said tumor suppressor function of p53 may be the result of one or more mutations in the p53 gene in combination with exposure to one or more metals, whereas in the absence of said one or more metals, but in the presence of said one or more mutations there is no impaired p53 function. More preferably said inactivation of said tumor suppressor function of p53 is mediated by one or more mutations in the p53 gene at normal or near normal levels of metals, wherein said tumor suppressor function may be restored by administration of a chelating agent.

In a medical use of the invention, the cancer can be selected from the group of carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; Preferably, leukemia is lymphoid leukemia; plasma cell leukemia; erythroleukemia; lymphosarcoma cell leukemia; myeloid leukemia; acute myeloid leukemia (AML), chronic myeloid leukemia (CML); basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); hairy cell leukemia; chronic myeloblastic leukemia; or acute lymphoblastic leukemia (ALL). In some embodiments, said cancer is a solid tumor. In some embodiments, said cancer is a liquid tumor. In preferred embodiments, said cancer is an acute myeloid leukemia (AML); a pancreatic cancer; a lung cancer such as small cell lung cancer (SCLC); a cancer of the gastrointestinal tract or breast cancer. More preferably, said cancer is AML, lung cancer such as SCLC or pancreatic cancer.

### Anti-Cancer Therapeutic Agent

The present invention relates to a chelating agent for use in a method of treating cancer in a subject, wherein said cancer has a resistance to an anti-cancer therapeutic agent. Said anti-cancer therapeutic agent can be any anti-cancer therapeutic agent.

The anti-cancer therapeutic agent as described herein can be an anti-cancer therapeutic agent used in a treatment type selected from the group of chemotherapy, targeted therapy such as immunotherapy, stem cell therapy, hormone therapy, radiation therapy and surgery, or a combination thereof; preferably, said treatment type is chemotherapy. Said anti-cancer therapeutic agent can be selected from the group comprising Cyclophosphamide, Mechlorethamine, Chlorambucil, Melphalan, Dacarbazine, Nitrosoureas, Temozolomide, Daunorubicin, Doxorubicin, Epirubicin, Idarubicin, Mitoxantrone, Valrubicin, Cabazitaxel, Larotaxel, Ortataxel, Tesetaxel, Paclitaxel, Docetaxel, Abraxane, Taxotere, Epothilone A, Epothilone B, Epothilone C, Epothilone D, Epothilone E, Epothilone F, Vorinostat, Romidepsin, Irinotecan, Topotecan, Etoposide, Teniposide, Tafluposide, Bortezomib, Erlotinib, Gefitinib, Imatinib, Vemurafenib, Vismodegib, Azacitidine, Azathioprine, Capecitabine, Cytarabine, Doxifluridine, Fluorouracil, Gemcitabine, Hydroxyurea, Mercaptopurine, Methotrexate, Tioguanine, Bleomycin, Actinomycin, Carboplatin, Cisplatin, Oxaliplatin, Nedaplatin, Triplatin Tetranitrate, Phenanthriplatin, Picoplatin, Satraplatin, Tretinoin, Alitretinoin, Bexarotene, Vinblastine, Vincristine, Vindesine, Vinflunine, Vinorelbine, Aminopterin, Pemetrexed, Pralatrexate, Raltitrexed, Pentostatin, Cladribine, Clofarabine, Fludarabine, Nelarabine, Carmofur, Floxuridine, Tegafur, Cytarabine, Gemcitabine, Decitabine, Hydroxycarbamide, Belotecan, Camptothecin, Cositecan, Etirinotecan pegol, Exatecan, Gimatecan, Lurtotecan, Rubitecan, Silatecan, Aclarubicin, Amrubicin, Pirarubicin, Valrubicin, Zorubicin, Mitoxantrone, Losoxantrone, Pixantrone, Bendamustine, Chlormethine, Ifosfamide, Trofosfamide, Prednimustine, Uramustine, Carmustine, Fotemustine, Lomustine Semustine, Nimustine, Ranimustine, Streptozocin, Mannosulfan, Treosulfan, Carboquone, Thiotepa, Triaziquone, Triethylenemelamine, Altretamine, Procarbazine, Mitobronitol, Pipobroman, Dacarbazine, Temozolomide, Dactinomycin, Bleomycin, Mitomycins, Plicamycin, Aminolevulinic acid, Efaproxiral, Methyl aminolevulinate, Padeliporfin, Porfimer sodium, Talaporfin, Temoporfin, Verteporfin, Tipifarnib, Abemaciclib, Alvocidib, Palbociclib, Ribociclib, Seliciclib, Bortezomib, Carfilzomib, Oprozomib, Ixazomib, Anagrelide, Tiazofurin, Masoprocol, Niraparib, Olaparib, Rucaparib, Belinostat, Entinostat, Panobinostat, Romidepsin, Vorinostat, Pi3K, Alpelisib, Copanlisib, Duvelisib, Idelalisib, Umbralisib, Atrasentan, Bexarotene, Testolactone, Amsacrine, Arsenic trioxide, Asparaginase, Pegaspargase, Belzutifan, Celecoxib, Demecolcine, Elesclomol, Elsamitrucin, Eribulin, Estramustine phosphate, Etoglucid, Lonidamine, Lucanthone, Mitoguazone, Mitotane, Oblimersen, Omacetaxine mepesuccinate, Trabectedin, Alitretinoin, Bexarotene, Tretinoin, Veliparib and Venetoclax. In some embodiments, a combination of the above-listed therapeutic agents is selected as the anti-cancer therapeutic agent as described herein. Preferably, said combination is a known chemotherapy regimen, such as CMF (Cyclophosphamide, Methotrexate, 5-fluorouracil, vinorelbine), AC (doxorubicin, cyclophosphamide), DA (cytarabine, an anthracycline antibiotic, daunorubicin), IA (cytarabine, an anthracycline antibiotic, idarubicin), DAT (daunorubicin, cytarabine, tioguanine), FLAMSA (fludarabine, cytarabine, amsacrine), FLAMSA-BU (fludarabine, cytarabine, amsacrine, busulfan), FLAMSA-MEL (fludarabine, cytarabine, amsacrine, melphalan), TAD (tioguanine, cytarabine, daunorubicin), CAF (cyclophosphamide, doxorubicin, fluorouracil), CLIA (cladribine, idarubicin, and cytarabine), CLIA-M (mylotarg, cladribine, idarubicin, and cytarabine) and ABVD (doxorubicin, bleomycin, vinblastine, dacarbazine). Preferably, an anthracycline such as a doxorubicin, an antimetabolite, such as 5-fluorouracil (5-FU), and/or a taxane, such as Nab-paclitaxel and/or Paclitaxel is selected as the anti-cancer therapeutic agent to which a cancer as described herein is resistant.

The anti-cancer therapeutic agent as disclosed herein can be administered by any acceptable delivery mode, such as e.g. by liposomal delivery.

### Administration

The present invention relates to a chelating agent for use in a method of treating cancer in a subject, wherein said cancer has a resistance to an anti-cancer therapeutic agent. Preferably, said method of treating cancer in a subject further comprises co-administering an anti-cancer therapeutic agent as described herein. Preferably, administration of said anti-cancer therapeutic agent is performed parenterally. Said parenteral administration can be epidural, intracerebral, intracerebroventricular, epicutaneous, sublingual, extra-amniotic, nasal, intra-arterial, intra-articular, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal or intratumoral administration; more preferably the administration is intravenous or intratumoral administration.

The administration of a chelating agent as disclosed herein is performed parenterally or enterally. Said parenteral administration can be epidural, intracerebral, intracerebroventricular, epicutaneous, sublingual, extra-amniotic, nasal, intra-arterial, intra-articular, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, rectal or intratumoral administration; more preferably said administration is intravenous or intratumoral administration or the administration is orally or rectally.

A second and optionally further chelating agent can be employed in a medical use of the invention. Preferably, said second chelating agent is administered together with the first chelating agent, e.g. together at the same time (such as in the form of a single pharmaceutical composition), separately of each other at the same time (for instance in the form of separate pharmaceutical compositions) or separately of each other staggered in time. Simultaneous, separate and sequential administration of chelating agents as disclosed herein in the same treatment schedule are expressly envisaged. Said second chelating agent can for instance be administered parenterally or enterally. More preferably, said parenteral administration is epidural, intracerebral, intracerebroventricular, epicutaneous, sublingual, extra-amniotic, nasal, intra-arterial, intra-articular, intracardiac, intracavernous, intradermal, intralesional, intramuscular, intraocular, intraosseous, intraperitoneal, intrathecal, intrauterine, intravaginal, intravenous, intravesical, intravitreal, subcutaneous, transdermal, perivascular, transmucosal, rectal or intratumoral administration; still more preferably the administration is intravenous or intratumoral administration, or is orally or rectally.

In some embodiments, a third, fourth, fifth or further chelating agent is used in a medical use of the invention. Preferably, said third, fourth, fifth or further chelating agent is administered together with the first and second chelating agent as described above.

In some embodiments, a chelating agent, and optionally a second, third, fourth, fifth and/or further chelating agent, is administered together with one or more anti-cancer therapeutic agents as described herein. In some embodiments the one or more chelating agents are solitarily administered, i.e., in absence of an anti-cancer therapeutic agent. For example, the one or more chelating agents are solitarily administered followed by administration of an anti-cancer therapeutic agent or by a combined administration of an anti-cancer therapeutic agent and one or more chelating agents. For example, the one or more chelating agents are solitarily administered, i.e., in absence of an anti-cancer therapeutic agent, for at least 10 minutes, at least 30 minutes, at least one hour, at least two hours, at least three hours, at least four hours, at least five hours, at least 6 hours, at least 8 hours, at least 12 hours, at least 24 hours, at least 48 hours or at least 72 hours prior and/or after administration of an anti-cancer therapeutic agent and/or of a combination of an anti-cancer therapeutic agent and said chelating agent. For examples the one or more chelating agents are solitarily administered for 10 minutes to 30 minutes, 10 minutes to 72 hours, 30 minutes-48 hours, 1 hour to 24 hours, 2 hours to 12 hours, or 1 hour to 3 hours prior and/or after administration of an anti-cancer therapeutic agent and/or of a combination of an anti-cancer therapeutic agent and said chelating agent. For example, the chelating agent is administered at least 10 minutes, 30 minutes, at least one hour, at least two hours, at least three hours, at least four hours, at least five hours, at least 6 hours, at least 8 hours, at least 12 hours or at least 24 hours prior and/or after to a first, a second, a third and/or every continuing administration cycle of said anti-cancer therapeutic agent or of an administration cycle of a combination of an anti-cancer therapeutic agent and one or more chelating agents. For example, the one or more chelating agents is administered at least 10 minutes, 30 minutes, at least one hour, at least two hours, at least three hours, at least four hours, at least five hours, at least 6 hours, at least 8 hours, at least 12 hours or at least 24 hours prior and/or after to every administration cycle of said anti-cancer therapeutic agent or of an administration cycle of a combination of an anti-cancer therapeutic agent and one or more chelating agents.

For example, the administration of one or more chelating agents in absence of an anti-cancer therapeutic agent (solitary administration) is repeated after administration of an anti-cancer therapeutic and/or after administration of a combination of one or more chelating agents and an anti-cancer therapeutic agent.

Preferably, the same route of administration is selected for a chelating agent, and optionally a second, third, fourth, fifth and/or further chelating agent, and one or more anti-cancer therapeutic agents as described herein. However, it is also possible that the chelating agent and said anti-cancer therapeutic agent are administered through different routes of administration. As an example, the anti-cancer therapeutic agent can be administered parenterally, and the chelating agent orally. Further, as an example, the (first) chelating agent can be administered orally, and the second, or further, chelating agent can be administered parenterally.

A chelating agent as disclosed herein is preferably in composition that further comprises a pharmaceutically acceptable excipient (or carrier). As used herein, pharmaceutically acceptable excipient or carrier includes reference to any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the pharmaceutical compositions is contemplated. Acceptable excipients, carriers or diluents for therapeutic use are well known in the pharmaceutical art, and are described, for example, in standard textbook references such as Remington's Pharmaceutical Sciences (e.g. Mack Publishing Co., A. R. Gennaro edit., 1985.

In a medical use of the invention, said chelating agent can be administered to a subject at least once, at least twice, at least three times, at least four times, at least five times, at least six times, at least seven times, at least eight times, at least nine times, at least ten times, at least twelve times, at least fourteen times, at least sixteen times, at least eighteen times, at least twenty times, at least twenty-five times, at least thirty times, at least thirty-five times, at least forty times, at least fifty times, at least sixty times, at least seventy times, at least eighty times, at least ninety times or at least one hundred times.

Preferably, a chelating agent as disclosed herein is employed in a treatment regimen that involves daily, weekly or monthly administration of said chelating agent. Preferably, treatment is maintained for at least three days, at least a week, at least a month, and more preferably at least 6 months or at least a year such as 2-5 years.

Preferably, a chelating agent as disclosed herein and/or an anti-cancer therapeutic agent is employed in a treatment regimen that involves administration in cycles where each cycle comprises repetitive administration, for example daily administration, of said chelating agent for several days, for example for at least one day, at least three days, at least a week, at least two weeks or at least three weeks. Preferably the administration of a chelating agent as disclosed herein and/or an anti-cancer therapeutic agent in cycles is repeated every two weeks, every three weeks, every four weeks, every five weeks, every six weeks, every seven weeks, every eight weeks. Preferably, the administration of a chelating agent as disclosed herein and/or an anti-cancer therapeutic agent in cycles is repeated after 3-5 weeks, for example after three weeks or after five weeks. Preferably, the administration of a chelating agent as disclosed herein and/or an anti-cancer therapeutic agent may be repeated at no fixed interval, but according to the patient's need. Preferably, the administration of a chelating agent as disclosed herein and/or an anti-cancer therapeutic agent in cycles is repeated at least one time, at least two times, at least thee three times, at least four times, at least five times, at least six times, at least seven times, at least eight times, or at least 10 times. Preferably, treatment is maintained for at least three days, at least a week, at least a month, and more preferably at least six months or at least a year, such as 2-5 years.

Administration of a chelating agent as disclosed herein or an anti-cancer therapeutic agent as disclosed herein to a subject may follow general protocols for the administration of such compounds, taking into account the toxicity, if any, of the agents. Therefore, in some embodiments there is a step of monitoring toxicity that is attributable to combination therapy.

A chelating agent as disclosed herein can be administered in any acceptable pharmaceutical dosage form, for example as an aqueous medium such as a solution, suspension, emulsification. A chelating agent can also be administered orally as a pill, tablet, capsule, etc.

In a medical use of the invention, the subject can be identified as eligible for therapy if he or she has a cancer with cancer cells that exhibit a resistance such as a chemoresistance as disclosed herein. Such patients may benefit from a medical use of the invention.

### NUMBERED EMBODIMENTS

Embodiment 1. A method of treating a subject having a cancer, wherein said cancer has a resistance to one or more anti-cancer therapeutic agents, said method comprising the steps of:
administering a therapeutically effective amount of a chelating agent to a subject having a cancer, wherein said cancer has a resistance to one or more anti-cancer therapeutic agents.

Embodiment 2. The method according to embodiment 1, wherein said method further comprises a step of administering a therapeutically effective amount of said one or more anti-cancer therapeutic agents.

Embodiment 3. The method according to embodiment 1 or embodiment 2, wherein said method of treating is a method of sensitizing a cancer of a subject to one or more anti-cancer therapeutic agents.

Embodiment 4. The method according to any one of the preceding embodiments, wherein said method of treating is a method of chemosensitizing a cancer of a subject.

Embodiment 5. The method according to any one of the preceding embodiments, wherein said method of treating is a method of potentiating an anti-cancer effect of said one or more anti-cancer therapeutic agents.

Embodiment 6. The method according to embodiment 5, wherein said anti-cancer effect that is potentiated is selected from the group consisting of a cytotoxic effect, a cytostatic effect, anti-invasiveness; anti-dissociation; anti-vascularization, and combinations thereof.

Embodiment 7. The method according to any one of the preceding embodiments, wherein said resistance to said one or more anti-cancer therapeutic agents is a metal-induced resistance, preferably wherein said metal is selected from the group consisting of iron, copper, lead, cadmium, mercury, chrome, vanadium (e.g. in the form of VO₄³⁻) and combinations thereof.

Embodiment 8. The method according to any one of the preceding embodiments, wherein said method further comprises a step of:
providing a sample of a subject having a cancer, preferably a sample comprising cancer cells;
identifying said subject as having a cancer that has a resistance to one or more anti-cancer therapeutic agents by measuring in said sample the presence of a p53-inactivating cancer cell metallome, preferably wherein said p53-inactivating cancer cell metallome is characterized by the presence of (an elevated level of) one or more metals selected from the group consisting of iron, copper, lead, cadmium, manganese, mercury, chrome, vanadium (e.g. in the form of VO₄³⁻) and combinations thereof, wherein said cancer has a resistance to one or more anti-cancer therapeutic agents.

Embodiment 9. The method according to any one of the preceding embodiments, wherein said one or more anti-cancer therapeutic agents is one or more chemotherapeutic agents.

Embodiment 10. The method according to any one of the preceding embodiments, wherein said one or more anti-cancer therapeutic agents is an anthracycline such as doxorubicin.

Embodiment 11. The method according to any one of the preceding embodiments, wherein said chelating agent is administered in combination with a second chelating agent;

Embodiment 12. The method according to any one of the preceding embodiments, wherein said chelating agent is a 2,3-dimercapto-1-propanesulfonic acid (DMPS) and optionally wherein said second chelating agent is an EDTA if a second chelating agent is present; or wherein said chelating agent is a 2,3-dimercaptosuccinic acid (DMSA), and optionally wherein said second chelating agent is an EDTA if a second chelating agent is present.

Embodiment 13. The method according to embodiment 11 or embodiment 12, wherein said chelating agent and optionally said second chelating agent are provided in the form of a fixed-dose product (preferably a fixed dose combination product), such as (i) a fixed-dose pharmaceutical composition comprising said chelating agent and optionally said second chelating agent or (ii) a fixed-dose kit comprising a first container that comprises said chelating agent and second container that comprises said second chelating agent.

Embodiment 14. The method according to any one of the preceding embodiments, wherein said chelating agent, and optionally said second chelating agent, are administered parenterally, such as intravenously or intratumorally, or enterally such as orally or rectally.

Embodiment 15. The method according to any one of the preceding embodiments, wherein said one or more anti-cancer therapeutic agent is administered parenterally, such as intravenously or intratumorally.

Embodiment 16. The method according to any one of the preceding embodiments, wherein said chelating agent, and optionally said second chelating agent, are administered in a dose of 1-100 mg/kg/day, daily for 1-25 days of each cycle, and provided in repeated cycles at intervals (e.g. intervals of 3-6 weeks apart).

Embodiment 17. The method according to any one of the preceding embodiments, wherein said cancer is a solid tumor or a liquid tumor.

Embodiment 18. The method according to any one of the preceding embodiments, wherein said cancer is a breast cancer, a lung cancer such as SCLC, a pancreatic cancer or a blood cancer such as AML.

Embodiment 19. A chelating agent for use in a method of treating a cancer in a subject, wherein said cancer has a resistance to an anti-cancer therapeutic agent.

Embodiment 20. The chelating agent for use according to embodiment 19, wherein said chelating agent is for use in a method of counteracting a resistance of said cancer to said anti-cancer therapeutic agent.

Embodiment 21. The chelating agent for use according to embodiment 19 or embodiment 20, wherein said chelating agent is for administration in combination with said anti-cancer therapeutic agent.

Embodiment 22. The chelating agent for use according to any one of the embodiments 19-21, wherein said resistance is a chemoresistance; and wherein said anti-cancer therapeutic agent is a chemotherapeutic agent.

Embodiment 23. The chelating agent for use according to any one of the embodiments 19-22, wherein cancer cells of said cancer have an impaired tumor suppressor protein function.

Embodiment 24. The chelating agent for use according to embodiment 23, wherein said impaired tumor suppressor protein function is the result of aberrant tumor suppressor protein folding.

Embodiment 25. The chelating agent for use according to any one of the embodiments 19-24, wherein cancer cells of said cancer comprise an aberrantly folded tumor suppressor protein resulting in impaired tumor suppressor protein function.

Embodiment 26. The chelating agent for use according to any one of embodiments 23-25, wherein said tumor suppressor protein is one or more tumor suppressor proteins selected from the group consisting of p53, p63 and p73.

Embodiment 27. The chelating agent for use according to any one of embodiments 23-26, wherein said tumor suppressor protein is p53; and preferably wherein said tumor suppressor protein function is one or more selected from the group formed by: negative regulation of the cell cycle and promotion of apoptosis.

Embodiment 28. The chelating agent for use according to embodiment 27, wherein p53 is a wild-type p53 or a mutated p53.

Embodiment 29. The chelating agent for use according to any one of embodiments 19-28, wherein said cancer is characterized by the presence of at least two, more preferably at least 3, 4, 5, 6, 7, 8, 9, 10, 11, 12, 13, 14, 15, 16, 17, 18, 19, 20, 21, 22 or at least 23 metals selected from the group consisting of arsenic (As), aluminum (Al), antimony (Sb), Barium (Ba), boron (B), cadmium (Cd), Cerium (Ce), Chromium (Cr), lead (Pb), mercury (Hg), neodymium (Nd), Manganese (Mn), Nickel (Ni), tin (Sn), titanium (Ti), uranium (U), vanadium (V), copper (Cu), iron (Fe), gold (Au), silver (Ag), palladium (Pd) and platinum (Pt).

Embodiment 30. The chelating agent for use according to any one of embodiments 19-29, wherein said cancer is characterized by the presence of elevated levels of at least two, more preferably at least 3, 4, 5, 6, 7, 8, 9, 10, 11, 12, 13, 14, 15, 16, 17, 18, 19, 20, 21, 22 or at least 23 metals selected from the group consisting of arsenic (As), aluminum (Al), antimony (Sb), Barium (Ba), boron (B), cadmium (Cd), Cerium (Ce), Chromium (Cr), lead (Pb), mercury (Hg), neodymium (Nd), Manganese (Mn), Nickel (Ni), tin (Sn), titanium (Ti), uranium (U), vanadium (V), copper (Cu), iron (Fe), gold (Au), silver (Ag), palladium (Pd) and platinum (Pt).

Embodiment 31. The chelating agent for use according to any one of embodiments 19-30, wherein said cancer is characterized by the presence of elevated levels of at least one, preferably at least two, more preferably at least 3, 4, 5, 6, 7, 8 or at least 9 metals selected from the group consisting of copper (Cu), iron (Fe), lead (Pb), mercury (Hg), cadmium (Cd), Nickel (Ni), arsenic (As), vanadium (V) and Chromium (Cr).

Embodiment 32. The chelating agent for use according to any one of embodiments 25-31, wherein said aberrant tumor suppressor protein folding is induced by elevated levels of metals as defined in any one of embodiments 23-31.

Embodiment 33. The chelating agent for use according to any one of the embodiments 19-32, wherein said anti-cancer therapeutic agent is an anthracycline such as doxorubicin.

Embodiment 34. The chelating agent for use according to any one of the embodiments 19-33, wherein said chelating agent is administered in combination with a second chelating agent.

Embodiment 35. The chelating agent for use according to any one of the embodiments 19-34,
(i) wherein said chelating agent is a 2,3-dimercapto-1-propanesulfonic acid (DMPS) and optionally wherein said second chelating agent, if present, is an EDTA; or
(ii) wherein said chelating agent is a 2,3-dimercaptosuccinic acid (DMSA), and optionally wherein said second chelating agent, if present, is an EDTA.

Embodiment 36. The chelating agent for use according to embodiment 34 or embodiment 35, wherein said chelating agent and optionally said second chelating agent are provided in the form of a fixed-dose product (preferably a fixed dose combination product), such as (i) a fixed-dose pharmaceutical composition comprising said chelating agent and optionally said second chelating agent or (ii) a fixed-dose kit comprising a first container that comprises said chelating agent and a second container that comprises said second chelating agent.

Embodiment 37. The chelating agent for use according to any one of the embodiments 19-36, wherein said chelating agent, and optionally said second chelating agent, are administered parenterally (such as intravenously or intratumorally) or enterally (such as orally or rectally).

Embodiment 38. The chelating agent for use according to any one of the embodiments 19-37, wherein said anti-cancer therapeutic agent is administered parenterally such as intravenously or intratumorally.

Embodiment 39. The chelating agent for use according to any one of the embodiments 19-38, wherein said chelating agent, and optionally said second chelating agent, are administered in a dose of 1-100 mg/kg/day, daily for 1-25 days of each cycle, and provided in repeated cycles at intervals (e.g. intervals typically 3-6 weeks apart).

Embodiments 40. The chelating agent for use according to any one of the embodiments 19-39, wherein said cancer is a solid tumor or a liquid tumor.

Embodiment 41. The chelating agent for use according to any one of the embodiments 19-40, wherein said cancer is a breast cancer, a lung cancer such as small cell lung cancer (SCLC), a pancreatic cancer or a blood cancer such as acute myeloid leukemia (AML).

Embodiment 42. A pharmaceutical composition comprising a 2,3-Dimercapto-1-propanesulfonic acid (DMPS) and a pharmaceutically acceptable excipient; wherein the DMPS is present in a dose of 40-12000 mg, preferably 400-3600 mg; preferably wherein said composition is for daily administration.

Embodiment 43. A pharmaceutical composition comprising (i) a 2,3-Dimercapto-1-propanesulfonic acid (DMPS) or a DMSA, (ii) an EDTA, and (iii) a pharmaceutically acceptable excipient; preferably wherein said DMPS or said DMSA is present in a dose of 40-12000 mg, preferably 400-3600 mg.

Embodiment 44. A pharmaceutical composition according to embodiment 42 or embodiment 43, further comprising a chemotherapeutic agent, preferably an anthracycline such as doxorubicin.

Embodiment 45. A pharmaceutical combination comprising (i) a first container comprising a pharmaceutical composition comprising a 2,3-Dimercapto-1-propanesulfonic acid (DMPS) or a DMSA, and a pharmaceutically acceptable excipient; and (ii) a second container comprising a pharmaceutical composition comprising a chemotherapeutic agent, preferably an anthracycline such as doxorubicin, and a pharmaceutically acceptable excipient; and optionally wherein said combination comprises a third container comprising an EDTA, and a pharmaceutically acceptable excipient.

### EXAMPLES

### Example 1

### Materials and Methods

### Cell Lines

A549, MCF7 and Beas-2B cells were obtained from ATCC (https://www.atcc.org). Cells were maintained in DMEM high Glucose (Invitrogen) and 10% FCS (Gibco) at 37° C. and 5% CO2. A549 p53 KO cells were generated using a CRISPR p53 KO construct (pLV-U6g-EPCG with target sequence TCCATTGCTTGG GACGGCAAGG, Sigma). Cell lines were selected for GFP expression using FACS sorting followed by clonal selection in neomycin (600 ng/ml, Sigma).

### Metals/Chelators and Chemotherapy Solutions

Metals were dissolved individually in 10 mM HNO₃ and combined in the desired ratio in a parent mixture which was sterilized by filtration. Small volumes of the parent mixture were added to the incubation media to provide different amounts of metals to the cells, as detailed in Table 1. An equivalent amount of NaOH was added to prevent pH fluctuations.

TABLE 1
Metal concentrations used in the experimental work

| | | | Metal mixtures for in vitro experiments | | | |
|---|---|---|---|---|---|---|
| | | | 1:256 | 1:128 | 1:64 | 1:32 |
| | | | Concentration | Concentration | Concentration | Concentration |
| Metal | Preferred salt | Name | (μM) | (μM) | (μM) | (μM) |
| Cr | K₂Cr₂O₇ | Potassium dichromate | 0.25 | 0.50 | 1.00 | 2.00 |
| Mn | MnCl₂ | Manganese(II) chloride tetrahydrate | 2.00 | 4.00 | 8.00 | 16.00 |
| Cu | CuSO₄ | Cupric sulfate pentahydrate | 2.50 | 5.00 | 10.00 | 20.00 |
| Zn | ZnCl₂/ZnSO₄ | Zinc chloride | 2.50 | 5.00 | 10.00 | 20.00 |
| Cd | CdCl₂ | Cadmium chloride hemipentahydrate | 0.30 | 0.60 | 1.20 | 2.40 |
| Hg | HgCl₂ | Mercury(II) chloride | 1.50 | 3.00 | 6.00 | 12.00 |
| Pb | (CH₃COO)₂Pb | Lead(II) acetate trihydrate | 1.00 | 2.00 | 4.00 | 8.00 |

DMPS stock solution was 1 mM in water/filtered and stored at −20° C. in aliquots, EDTA was dissolved as a 0.5M solution in water in which pH was corrected to 8 with 2M NaOH/filtered and stored at RT.

Doxorubicin was made as a 32 mM solution in water/filtered and stored in aliquots at −20° C.

### Cell Survival Assays

Resazurin cell survival assays were used to determine cell survival upon chemotherapeutic and chelator challenge. 2×10³ cells were seeded in 100 μl in 96 well plates. 24 hrs later medium was replaced with 90 μl of metal mixtures or control medium for another 24 hrs. Next a combination of chelators and chemotherapy as indicated in the figures was prepared and added as an additional 10 μl to the cells. Cells were then incubated for 72 hr (Beas-2B) or 96 hr (A549 and MCF7). A stock solution of resazurin (Sigma) was made as 880 μM in PBS adjusted to pH 7.8/filtered 0.2 μm and kept for 6 months at 4° C. Medium was replaced with 75 μl of resazurin solution (final concentration 44 μM). Cells were incubated at 37° C. for 3-4 hrs and fluorescence emission was measured at 583 nm with excitation at 555 nm using a spectrophotometer (Beckman). To test the effects of metals on doxorubicin directly, doxorubicin was incubated with metals in solution 24 hrs as a 10× solution, prior to adding to cells, whereas control cells were incubated in metals as described before.

### P53 Folding/Western Blot

Cells were seeded in 6 well plates at 30% cell density. 24 hrs later, medium was replaced with the metal mixtures as indicated. Cells were incubated for 24 hrs and lysed with 100 μl NP-40 lysis buffer (100 mM NaCl 100 mM Tris pH8, 1% NP-40) for 15 min on ice. Cell pellets were discarded in centrifugation (10 min max) and 10% of input was taken for western blot and mixed with 4×SB. The remaining lysate was subjected to immunoprecipitation with p53 Ab240 Ab (1 ul per condition) using 30 μl NP-40 buffer washed goat-anti-mouse magnetic Dynabeads (Thermo Fisher). Lysates and beads were tumbled at 4° C. for 2 hrs and washed 3× with NO-40 buffer. Beads were taken up in 2×SB and together with inputs denatured at 95° C. in a heat block. 8% SDS page gels were used to run all of the samples on western blot. Gels were transferred on nitrocellulose membrane, blocked with milk (TBS-Tween) for 1 hr tumbling and incubated in p53 DO-1 (Santa Cruz Ab, 1:2000) ON at 4° C. The membrane was washed 3× with TBS-tween and secondary Ab (anti-mouse 700, Li-Cor) was used 1:10.000 to incubate the blot for 1 hr at room temperature. The blot was washed with TBS-tween 3× and the signal was measured in a Li-Cor Odyssey. Quantification was measured in the software of Li-Cor and plotted corrected for input levels of each sample.

### Doxorubicin Uptake Assay

1×10⁴ Cells were seeded in 96 wells for 24 hrs. Cells were then incubated with the metal mixture 1:128 for 24 hrs. 10% volume of doxorubicin at indicated concentrations was subsequently added and doxorubicin accumulation was measured 2 hrs later using the Opera Phenix high-content imaging screening system with 564 nm excitation. Representative images for each concentration are shown.

### Results

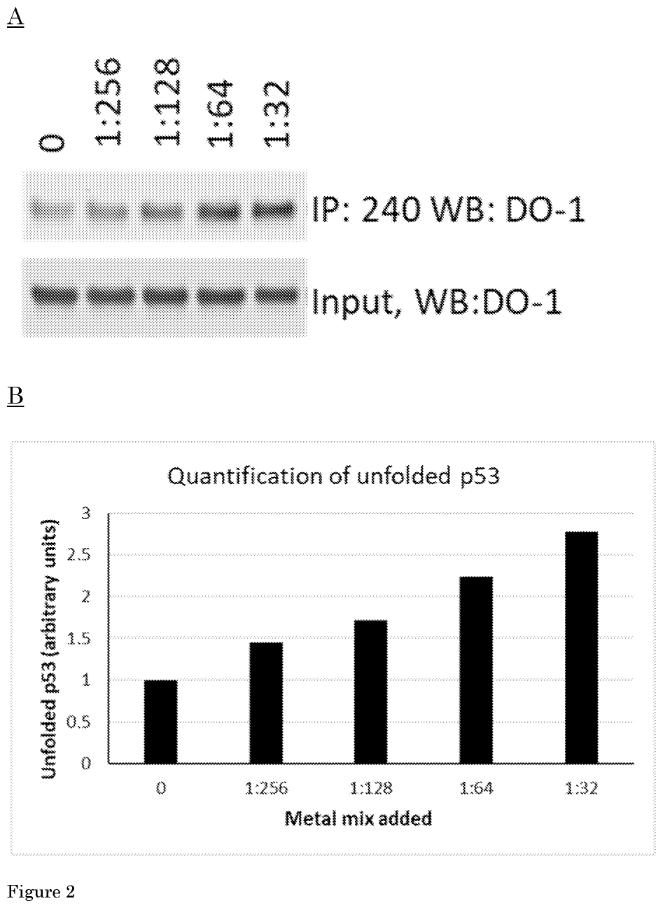

In order to test if metals convey chemoresistance, an immortalised Beas-2B lung cell line was exposed to increasing doses of a metal mixture for 24 hrs. Cells were subsequently subjected to a concentration range of doxorubicin and survival was measured after 72 hrs using resazurin survival assays (FIG. 1). It is clear that increasing doses of metals increase resistance to doxorubicin in a dose-dependent manner. In a concomitant assay using similar concentrations of metals, we determined p53 expression and folding status. These assays showed that p53 expression levels do not change upon metal exposure, but that a higher proportion of p53 is in an unfolded state upon increased metal levels (FIGS. 2A and B).

We next tested if the chemoresistance caused by metals could be reversed by adding a high dose (150 μM) of the chelator DMPS to Beas2B cells. In cells primed with a 1:128 and a 1:256 dilution of metals, sensitivity to doxorubicin was completely restored when DMPS was added concomitantly with the metals (FIG. 3A). Similar results were observed in the breast cancer cell line MCF7 (FIG. 3B) and in the adenocarcinoma lung cancer cell line A549 (FIG. 3C), indicating this effect can be observed in multiple different cancer cells. Most interestingly, A549 p53 KO cells exposed to metals developed only weak chemoresistance and chelators were not able to restore sensitivity to the same extent as in A549 control cells, although it has to be noted that overall growth of these cells was lacking compared to control A549 cells (FIG. 3D).

EDTA was used at the same chelation capacity as DPMS. EDTA is a multidentate ligand that forms 1:1 complexes with the added metals, whereas DMPS being bidentate will form complexes of varying stochiometry 1:1, 1:2 or 1:3. The most common is 1:2 which has been used as guiding principle in the calculation of equivalent chelation capacity. Thus, the molar concentration of EDTA (e.g. 75 μM) was half that of DMPS (e.g. 150 μM) to provide the same chelation capacity. EDTA at 75 μM was also able to restore chemosensitivity although not to the extent as seen for DMPS used at 150 μM (FIG. 4). We could not observe an additive effect of EDTA given in conjunction with DMPS. However, given that DMPS alone already almost completely restored doxorubicin sensitivity of the cells to the levels seen in the absence of metals, combined effects had to be tested at lower doses of chelators. We therefore lowered DMPS to 40 μM and EDTA to 20 μM. Each individual dose of chelator at these levels was not able to restore chemosensitivity substantially. However, in combination they clearly reinforced each other in reversing the resistance to doxorubicin (FIG. 5).

In order to quantify the chemoresistance induced by metals, Beas-2B cells were incubated with or without metals and with EDTA, DMPS and their combinations and exposed to doxorubicin. IC₅₀—the doxorubicin concentration that would kill 50% of the cells—was calculated (FIG. 6). It is seen that without added metals the IC₅₀ of doxorubicin was low and not affected by chelators whereas in cells that had been loaded with metals (1:128) it was more than 10 times higher. This resistance was largely reversed by chelator treatment.

Previous research had determined that doxorubicin could complex with certain metals, which in breast cancer enhanced its activity (Jabłońska-Trypuć et al., Molecules, 22 (7): 1106 (2017)). This is in possible contradiction to what we observed, but it did make us wonder if our metal mixture complexed with doxorubicin, preventing its uptake. As doxorubicin is fluorescent, we did a fluorescent uptake assay in which Beas-2B cells were loaded with a 1:128 dilution of metal mix prior to being exposed to increasing doses of doxorubicin for 2 hrs. No difference was observed in the amount of doxorubicin uptake between metal-exposed and control cells (FIG. 7). In addition, we also tested if pre-incubating doxorubicin with metals prior to adding it to the cells incapacitated the doxorubicin. Pre-mixing metals with doxorubicin did not prevent doxorubicin from causing cell death. Sensitivity to doxorubicin in this condition was better than in metal primed cells, but worse than in cells that had not seen any metals (FIG. 8). Moreover, it was evident that preincubating cells with metal before doxorubicin exposure, facilitating metal uptake, was effective in inducing chemoresistance whereas simultaneous administration of metals and doxorubicin was much less effective, suggesting that metal uptake is required for induction of chemoresistance.

Together, these data suggest that metals induce resistance to doxorubicin in multiple cancer cell lines. Metal induced chemoresistance seems largely to be p53 dependent and coincides with a dose-dependent unfolding of p53. Finally, chelators are able to fully restore doxorubicin sensitivity. Our data suggest that metals drives a yet unknown intracellular chemoresistance phenotype that is dependent on p53 signalling.

### Example 2

### Materials and Methods

Overall, the methods disclosed in the first example were used in the second example as well. Additionally, the following methods were used:

### Additional Cells:

Hep3B and H2177 cells were obtained from ATCC and were maintained in DMEM high Glucose (Invitrogen) and 10% FCS (Gibco) at 37° C. and 5% CO2. Beas2B cells were plated in 6 well plates and transfected the following day with 1.5 μl of 20 μM of p53 siRNA (pool of 4 from Dharmacon) or a control siRNA (pool of 4 from Dharmacon) using 4.5 μl lipofectamine and 500 μl serum free medium following (Thermo Fisher Scientific). Cells were subsequently counted and added to 96 well plates for survival assays as previously described. Hep3B cells were transfected with p53 plasmids (described in Muller et al, Cell 2009) using lipofectamine 3000 (Thermo Fisher Scientific).

### Folding Experiments In Vitro:

In order to assess direct effects of metals and chelators on p53 folding, Beas-2B cells were lysed and lysates treated with metals for 30 min on ice in NP40 buffer. If chelators were used, these were subsequently added for 1 hr on ice. Lysates were then subjected to IP conditions as described previously.

### Data Analysis

IC50 values were calculated using Graphpad Prism. Error bars represent standard deviation of triplicate measurements.

### Metals/Chelators and Chemotherapy Solutions

Solutions were prepared as previously described. Different amounts of metals were provided to cells, as detailed in Tables 2 and 3.

TABLE 2
Metal concentrations used for MCF7 and Hec-1a cells

| | | | Metals for MCF7 and Hec-1a cells | | |
|---|---|---|---|---|---|
| | | | 1:256 | 1:128 | |
| | | | Concentration | Concentration | |
| Metal | Preferred salt | Name | (μM) | (μM) | Comments |
| V | Na₃VO₄ | Sodium orthovanadate | 0.40 | 0.80 | |
| Cr | K₂Cr₂O₇ | Potassium dichromate | 0.16 | 0.32 | |
| Mn | MnCl₂ | Manganese(II) chloride tetrahydrate | 4.00 | 8.00 | |
| Fe | (NH₄)_{y}Feₓ(C₆H₈O₇) | Ammonium iron(III) citrate | 4.00 | 8.00 | |
| Co | CoCl₂ | Cobalt(II) chloride hexahydrate | 3.25 | 6.50 | |
| Cu | CuSO₄ | Cupric sulfate pentahydrate | 2.00 | 4.00 | |
| Zn | ZnCl₂/ZnSO₄ | Zinc chloride | 5.00 | 10.00 | |
| As | NaAsO₂ | Sodium (meta)arsenite | 1.25 | 2.50 | Prepare fresh |
| Se | Na₂SeO₃ | Sodium selenite | 0.80 | 1.60 | |
| Rb | RbCl | Rubidium chloride | 30.00 | 60.00 | |
| Cd | CdCl₂ | Cadmium chloride hemipentahydrate | 0.33 | 0.65 | |
| Hg | HgCl₂ | Mercury(II) chloride | 1.60 | 3.20 | Prepare fresh |
| Pb | (CH₃COO)₂Pb | Lead(II) acetate trihydrate | 0.33 | 0.65 | |

TABLE 3
Metal concentrations used for Beas-2B cells

| Metals for Beas-2B cells | | | | | |
|---|---|---|---|---|---|
| | | | Metal Mix 1:256 | Metal Mix 1:128 | |
| Metal | Preferred salt | Name | Conc. (μM) | Conc. (μM) | Comments |
| V | Na₃VO₄ | Sodium orthovanadate | 0.17 | 0.34 | |
| Cr | K₂Cr₂O₇ | Potassium dichromate | 0.21 | 0.43 | |
| Fe | (NH₄)_{y}Feₓ(C₆H₈O₇) | Ammonium iron(III) citrate | 1.70 | 3.40 | Prepare fresh |
| Co | CoCl₂ | Cobalt(II) chloride hexahydrate | 1.50 | 3.00 | |
| Ni | NiCl₂ | Nickel(II) chloride | 4.25 | 8.50 | |
| Cu | CuSO₄ | Cupric sulfate pentahydrate | 2.13 | 4.25 | |
| Zn | ZnCl₂/ZnSO₄ | Zinc chloride | 2.13 | 4.25 | |
| As | NaAsO₂ | Sodium (meta)arsenite | 0.85 | 1.70 | Prepare fresh |
| Cd | CdCl₂ | Cadmium chloride hemipentahydrate | 0.26 | 0.51 | |
| Hg | HgCl₂ | Mercury(II) chloride | 1.28 | 2.55 | Prepare fresh |
| Pb | (CH₃COO)₂Pb | Lead(II) acetate trihydrate | 0.85 | 1.70 | |

### Cell Preparation for Metal Uptake Assessment

Cell cultures were grown according to suppliers (ATCC) instructions and incubated with metal mixtures as described for 24 hours.

At the end of experiments, cells were harvested by incubation with trypsin without EDTA for 8 min, collected and washed 3 times in phosphate buffered saline (PBS). Cells were centrifuged to create a pellet and transferred to metal assessment. In a high level cleanroom, 200 μL of pure HNO₃ (15N) and 100 μL of 30% H₂O₂ were added to the cell pellet, mixed well and incubated at room temperature for 2 hours, whereafter 1180 μL of double distilled water was added. The finished samples were transferred for metal measurement.

### ICP-MS Measurement

Following dilution, metal concentrations were determined using a quadrupole Inductively Coupled Plasma-Mass Spectrometer (Agilent Technologies, 8900 ICP-MS Triple Quad). The ICP-MS was calibrated with a series of multielement standard solutions (Merck; ME VI) and a blank. Drift was corrected by internal standards (750 μg/L Sc, 100 μg/L Re and 50 μg/L Rh).

### Results

The results of Example 1 showed that metal uptake is essential for the full occurrence of chemoresistance (FIG. 8). In order to determine the uptake of metals into the cells and the distinct effect of different metal mix concentrations added to the medium, cells were incubated with different concentrations of an extended metal mix for 24 hours in medium, washed and collected for ICP-MS assessment. In three different cells lines metals were taken up in a dose dependent fashion (FIG. 9, representative metals shown). This finding correlates well with the dependence of chemoresistance on metal uptake, as increased levels of added metals result in higher intracellular metal levels and in turn in increased chemoresistance.

In addition to unfolding already seen in Beas2B cells (FIG. 2), unfolding of p53 is shown in vitro in cell lysates exposed to low amounts of the metal mix (FIG. 10) and in other lung cancer cells, H2170, that have a 157 mutation in p53 which is very frequently found in small cell lung cancer and is, under normal conditions, present in a folded state (FIG. 11). Hep3B cells (p53 null) were used as a p53 null cell line sensitive to metal treatment to determine if certain mutants are more sensitive than the WT p53 to metal induced unfolding (FIG. 12). The 175H mutant is an unfolded mutant that is most prevalent in all cancers. As 175H p53 is already unfolded, no difference in folding can be detected upon metal addition. Mutants 157 and 158, in contrast, are more folded; yet it is clear that the ratio of unfolded to folded p53 is higher in response to lower metal concentrations in the 157 and 158 mutations than in WT p53. In other words, these mutants are more prone to unfold than WT p53, making such mutants an attractive target for chelation strategies.

Extending these findings further, it is herewith demonstrated that metals act in a p53 dependent manner to induce chemoresistance. Example 1 showed this in A549 cells using CRISPR KO lines (FIG. 3). In Example 2 a similar effect in Beas2B cells is demonstrated where p53 is knocked down which results in a clearly reduced IC50 in the p53 knockdown cells compared to controls (FIG. 13).

In Example 1 it is shown that EDTA and DMPS can reverse metal-induced resistance to cancer treatment in doxorubicin treated Beas-2B cells (FIGS. 3-5). In the current example it is demonstrated that miaDMSA (FIG. 14) and DMSA (FIG. 15) also reverse the metal-induced chemoresistance, indicating the efficacy of different types of chelators.

Importantly, chelators could also restore chemosensitivity to doxorubicin in a different more aggressive lung cancer cell line H2170 (FIG. 16). Other chemotherapeutics have also been examined, an example of which is shown in FIG. 17 where chemosensitivity of BxPC-3 to 5-FU was reduced by metals and restored with DMSA and miaDMSA, whereas BxPC-3 resistance to nab-paclitaxel in response to metals was more modest but nevertheless reversed by chelators (FIG. 18). In contrast, SW1990 cells did not show metal induced resistance against any chemotherapeutic (example in FIG. 19). It is important to note that BxPC-3 cells have a mutant version of p53-Y220 C—that has been shown to be refoldable, whereas SW1990 cells do not have any p53. Together, these findings support the notion that metals may induce resistance to various chemotherapeutics, whereas chelators can restore sensitivity.

Given the ability of chelators to prevent metal-induced chemoresistance or restore chemosensitivity, it was examined whether chelators could prevent unfolding of p53 and even restore folding once unfolding had occurred. Thus, Beas-2B cells were incubated with metals in the presence or absence of DMPS (FIG. 20) or DMSA (FIG. 21). DMPS and DMSA clearly prevented metal induced unfolding of p53. In a mutant setting in Hep3B cells, miaDMSA was able to prevent unfolding of 158 mutant p53 and WT p53 when exposed to metals (FIG. 22). To determine if any of these chelators were capable of refolding p53, we first incubated lysates from Beas-2B cells with metals after which we incubated them with chelators. miaDMSA and EDTA were able to refold p53 (FIG. 23).

### Discussion

Multiple different cell lines, different chemotherapy as well as different chelators have been added show that chelation strategies in combination with chemotherapy work in cells that have a WT p53 or a mutant version of p53 that is susceptible to refolding.

The induction of resistance to cancer therapy by a mixture of metals has been demonstrated in a number of cell lines, including Beas-2B bronchial epithelial cells (FIG. 1; FIG. 2A), MCF7 breast cancer cells (FIG. 2B), A549 lung cancer cells (FIG. 2C), H2170 squamous cell lung carcinoma (FIG. 16), BxPC3 epithelial pancreatic adenocarcinoma cells (FIG. 17-18) and to a certain extent in SW1990 pancreatic cells (FIG. 19). It is thus concluded that the metal induced resistance to therapy is a general feature of cancers.

In addition to unfolding of p53 by addition of metals seen in Beas-2B cells (FIG. 2), unfolding of p53 by addition of metals was also demonstrated in H2170 lung cancer cells and Hep3B hepatoma cells (FIG. 11-12). Unfolding of p53 was also demonstrated in vitro on lysates of cells exposed to low amounts of the metal mix (FIGS. 10 and 23), confirming that the unfolding of p53 is a direct effect of the exposure of the protein to the metal mix. Further, it is demonstrated that certain variants of mutant p53 that are folded under normal conditions may be more readily unfolded by the addition of metals than WT p53. Thus, folding and unfolding patterns were explored in lung cancer cells H2170 and hepatoma Hep3B cells. H2170 have a R158G mutation in p53, which is a very frequent mutation in small cell lung cancer, and its p53 is present in a folded state under normal conditions (FIG. 11). Hep3B cells (p53 null) were used as a p53 null cell line to determine if certain mutants are more sensitive than the WT p53 to metal induced unfolding (FIG. 12). The R175H mutant is an unfolded mutant that is most prevalent in all cancers. Mutants R157L and R158G in contrast are more folded and are enriched in SCLC. As R175H is already unfolded no metal-induced difference can be detected with this mutant. However, it is clear that in mutants R157L and R158G the ratio of unfolded to folded p53 is higher than in WT p53, even in response to much lower metal concentrations. In other words, these mutants are more prone to unfolding than WT p53 making such mutants an attractive target for chelation strategies in cancer.

Importantly, it is demonstrated that metals act in a p53 dependent manner to induce chemoresistance in cells. Thus, in a CRISPR knockout (KO) line of A549 cells, addition of metals had only a minor effect on cell viability when exposed to doxorubicin (FIG. 2D). Furthermore, metal exposure of p53 knockdown Beas-2B cells caused a much smaller increase in IC50 than in native Beas-2B cells (FIG. 13). These observations are further supported by the SW1990 pancreatic adenocarcinoma cell line, which does not have any p53 and did not show metal induced resistance against any chemotherapeutic (FIG. 19).

All chelators tested had a clear effect on cell viability when metal loaded cells were exposed to doxorubicin (FIGS. 3-6; 14-15) indicating that this strategy works with different types of chelators. It was demonstrated that especially miaDMSA (FIG. 14) is a highly potent chelator.

The ability of chelators to restore sensitivity to doxorubicin was observed across different cell lines. Thus, chelators could also restore chemosensitivity to doxorubicin in Beas-2B, A549, MCF7 (FIG. 3) and in a different more aggressive lung cancer cell line, H2170 (FIG. 16). While doxorubicin has been used extensively to demonstrate the effect of metals and chelators on p53 folding and chemoresistance, other chemotherapeutics have shown a similar pattern. As an example, the chemosensitivity of BxPC-3 to 5-fluorouracil (5-FU) was restored with DMSA and miaDMSA (FIG. 17). Interestingly, exposing BxPC-3 cells to nab-paclitaxel did not result in metal-induced resistance to this chemotherapeutic, but addition of miaDMSA or DMSA did reduce survivability. These data could mean that endogenous metal levels in these cells are sufficient to promote resistance to nab-paclitaxel (FIG. 18). Notably, BxPC-3 cells have a mutant version of p53 Y220C that has been shown to be refoldable. Together these findings support the notion that various chelators can restore sensitivity to various chemotherapeutics in a p53 dependent manner.

DMPS (FIG. 20) and DMSA (FIG. 21) clearly prevented metal induced unfolding of p53 in Beas-2B cells. In a mutant setting in Hep3B cells, miaDMSA was able to prevent unfolding of R158G mutant p53 and WT p53 when exposed to metals (FIG. 22). These results provide further evidence for the dependence of p53 unfolding on metals in a form that may be taken up by cells.

A consistent model of metal induced chemoresistance through a p53 unfolding mechanism requires that restoration of chemosensitivity is produced by refolding p53 or by cellular production of new, properly folded p53. To determine if any of these chelators were capable of refolding p53, lysates of Beas-2B cells were exposed to metals after which they incubated them with chelators. It was shown that miaDMSA and EDTA were able to refold p53 (FIG. 23) without a requirement for de novo synthesis of properly folded p53.

As proof of intracellular metal accumulation Beas-2B cells were exposed to metals for 24 hours, washed to eliminate all traces of added metals and collected for metal assessment by ICP-MS. A dose dependent metal uptake into cells is observed (FIG. 9). Similar results were obtained in other cell lines such as MCF7 and A549.

Altogether, data herein provided confirm the mechanism of action as an interplay between p53 and metals and further show a preference for the use of miaDMSA as a chelator to restore sensitivity to chemotherapy and to restore both WT p53 and mutant p53 folding.

1. A method of treating a cancer in a subject characterized with cancer cells having aberrant tumor suppressor protein folding resulting from presence of one or more metals, the method comprising,
administering a chelating agent to the subject, wherein the chelating agent chelates the one or more metals resulting in restored or re-activated tumor suppressor protein function, and
administering an anti-cancer therapeutic agent to the subject.

2. (canceled)

3. The method of claim 1, wherein said chelating agent is administered in combination with said anti-cancer therapeutic agent.

4. The method of claim 3, wherein said anti-cancer therapeutic agent is a chemotherapeutic agent.

5. The method of claim 1, wherein said cancer cells have an impaired tumor suppressor protein function resulting in resistance to the anti-cancer therapeutic agent.

6. (canceled)

7. (canceled)

8. The method of claim 1, wherein said tumor suppressor protein comprises one or more of p53, p63 and p73.

9. The method of claim 8, wherein said tumor suppressor protein comprises p53.

10. The method of claim 9, wherein p53 is a wild-type p53 or a mutated p53.

11. (canceled)

12. The method of claim 1, wherein the chelating agent is administered prior to administration of said anti-cancer therapeutic agent.

13. The method of claim 1, wherein the one or more metals comprises one or more of arsenic (As), aluminum (Al), antimony (Sb), Barium (Ba), boron (B), cadmium (Cd), Cerium (Ce), Chromium (Cr), lead (Pb), mercury (Hg), neodymium (Nd), Manganese (Mn), Nickel (Ni), tin (Sn), titanium (Ti), uranium (U), vanadium (V), copper (Cu), iron (Fe), gold (Au), silver (Ag), palladium (Pd) or platinum (Pt).

14. The method of claim 1, wherein the one or more metals is characterized by the presence of elevated levels of at least one or more of arsenic (As), aluminum (Al), antimony (Sb), Barium (Ba), boron (B), cadmium (Cd), Cerium (Ce), Chromium (Cr), lead (Pb), mercury (Hg), neodymium (Nd), Manganese (Mn), Nickel (Ni), tin (Sn), titanium (Ti), uranium (U), vanadium (V), copper (Cu), iron (Fe), gold (Au), silver (Ag), palladium (Pd) or platinum (Pt).

15. The method of claim 1, wherein the one or more metals is characterized by the presence of elevated levels of one or more of copper (Cu), iron (Fe), lead (Pb), mercury (Hg), cadmium (Cd), Nickel (Ni), arsenic (As), vanadium (V) or Chromium (Cr).

16.-18. (canceled)

19. The method of claim 12
wherein the chelating agent is administered at least 24 hours prior to administration of said anti-cancer therapeutic agent to restore or re-activate tumor suppressor protein function.

20.

21. The method of claim 1, wherein said anti-cancer therapeutic agent comprises an anthracycline, an antimetabolite, and/or a taxane.

22. The method of claim 1, wherein said chelating agent is administered in combination with a second chelating agent.

23. The method of claim 1, wherein the chelating agent comprises monoisoamylDMSA (miaDMSA).

24. The method of claim 1 wherein, said chelating agent comprises
2,3-dimercapto-1-propanesulfonic acid (DMPS) or
2,3-dimercaptosuccinic acid (DMSA) and optionally a second chelating agent.

25. The method of claim 1, wherein said chelating agent is administererd as a fixed-dose pharmaceutical composition comprising said chelating agent and optionally a second chelating agent.

26. The method of claim 1, wherein said chelating agent, and optionally a second chelating agent, are administered parenterally or enterally.

27. The method of claim 1, wherein said anti-cancer therapeutic agent is administered parenterally.

28. The method of claim 1, wherein said chelating agent, and optionally a second chelating agent, are administered in a dose of 1-100 mg/kg/day, daily for 1-25 days of a cycle, and provided in repeated cycles at intervals.

29. The method of claim 1, wherein said cancer is a solid tumor or a liquid tumor.

30. The method of claim 1, wherein said cancer is comprises a breast cancer, a lung cancer, a pancreatic cancer or a blood cancer.

31.-34. (canceled)

35. The method of claim 8 wherein said tumor suppressor protein function comprises negative regulation of the cell cycle or promotion of apoptosis.

36. The method of claim 12 wherein the chelating agent is administered at least 48 hours prior to administration of said anti-cancer therapeutic agent to restore or re-activate tumor suppressor protein function.
